SEQUENCE LISTING (1 sequence)

SEQ ID NO: 1 - protein, 10 aa, Human Immunodeficiency Virus-1
RGPGRAFVTI

(19) United States
(12) Patent Application Publication
(10) Pub. No.: US 2007/0298051 A1
(43) Pub. Date: Dec. 27, 2007

(54) Adjuvants Of Immune Response

(75) Inventors: Dan H Barouch, Boston, MA (US); Shawn M. Sumida, Honolulu, HI (US); Norman L. Letvin, Newton, MA (US)

Correspondence Address: CLARK & ELBING LLP, 101 FEDERAL STREET, BOSTON, MA, 02110, US

(73) Assignee: BETH ISRAEL DEACONESS MEDICAL CENTER, Boston, MA (US)

(21) Appl. No.: 10/580,105

(22) PCT Filed: Nov. 19, 2004
(86) PCT No.: PCT/US04/38865

§ 371 Date: May 2, 2007

Related U.S. Application Data
(60) Provisional application No. 60/523,380, filed on Nov. 19, 2003.

(51) Int. Cl. A61K 48/00; A61K 31/7088; A61K 38/02; A61P 31/00; A61P 33/00; A61P 37/06; A61P 37/08; A61P 35/00; A61P 31/18; A61K 38/45; A61K 39/12

(52) U.S. Cl. 424/199.1; 424/93.2; 424/94.5; 514/12; 514/44

(57) ABSTRACT

The present invention features methods to substantially increase the immunogenicity of a vaccine, preferably a DNA vaccine, and involves providing a mammal with a vaccine regimen, which includes an immunogen and Flt3L in combination with MIP-1α or MIP-3α. The methods of the present invention can be used for the prevention and treatment of various pathological states, including for example, cancer, microbial infections, autoimmune diseases, tissue rejection, and allergic reactions.

### STATEMENT AS TO FEDERALLY SPONSORED RESEARCH

This research was sponsored in part by NIH grant numbers AI-51223 and AI-58727. The government may have certain rights in the invention.

### FIELD OF INVENTION

The invention relates to the treatment, prevention, or reduction of pathological states by the use of vaccine preparations having greatly improved immunogenicity.

### BACKGROUND OF THE INVENTION

Vaccines are used for the prevention of infectious diseases as well as for the treatment and/or prevention of other pathological states, including cancer and autoimmune diseases. One of the long-standing goals in the field of vaccine development has been to substantially boost the immune response of the vaccinated mammal. Recent strategies for improving vaccines have focused on inducing a cellular immune response rather than only a humoral response. In the case of HIV infection for example, T cell responses play a pivotal role in controlling viral replication, and consequently, an effective AIDS vaccine will likely need to elicit a potent virus-specific cellular immune response.

Within the T cell repertoire, CD8+ T cells, or cytolytic T cells (CTLs), directly combat infections by lysing infected or ‘foreign’ cells and by suppressing proviral expression through the release of antiviral cytokines, such as tumor necrosis factors (TNFs) and interferon-γ. CD4+ T cells or helper T cells (Ths) further complement CD8+ T cells by providing growth factors and co-stimulatory molecules supporting the activation and maintenance of CD8+ T cells. The augmentation of a potent cellular response will therefore require vaccines to elicit a robust virus specific CD4+ and CD8+ T cell response.

It has been recognized that vaccines that have the ability to produce the target antigen in the cells of the vaccinated mammal are more effective in inducing a cellular response. Accordingly, sub-unit vaccines, which primarily include proteins and killed or inactivated vaccines, tend to only induce a humoral response. In contrast, live attenuated vaccines, recombinant vectors, and DNA vaccines, all of which lead to the production of antigens within the cells of the vaccinated mammal, induce a cellular response. Plasmid DNA vaccines, in particular, elicit CD8+ and CD4+ T cell responses as well as humoral responses in animal models. The possibility of developing DNA vaccines has therefore been an area of active investigation.

DNA vaccines have been shown to elicit immune responses to a diverse array of antigens, but their immunogenicity has proven quite limited. High doses of DNA vaccines are typically required to elicit potent immune responses in mice, and the immunogenicity of DNA vaccines in humans has been marginal to date. The mechanism of immune priming and the factors that limit the immunogenicity of DNA vaccines continue to remain poorly characterized.

Following intramuscular injection of a plasmid DNA vaccine in mice, expression of the encoded antigen occurs primarily in transfected myocytes at the site of inoculation. Myocytes lack expression of MHC class II and costimulatory molecules and thus would not be expected to prime T lymphocytes directly. Although it remains unclear, immune priming may occur by DCs. DCs are thought to present antigen by cross-presentation of extracellular antigen or following direct transfection of plasmid DNA. The DCs in these nonspecific inflammatory infiltrates, however, are only found in small numbers and typically exhibit functionally immature phenotypes. Consequently, the presentation of vaccine-derived antigen to the immune system is an inefficient process.

Novel and practical strategies to induce strong cellular responses are urgently needed to improve the efficiency of vaccines to control pathogenic states.

### SUMMARY OF THE INVENTION

We have discovered new methods for treating and preventing pathological states by substantially enhancing the immune response of a mammal (e.g., human) to a vaccine. The present invention is based on our discovery of the unexpected immunogenicity that results from the co-administration of an immunogen with a specific combination of adjuvants. Accordingly, the mammal of the invention is administered with a vaccine formulation containing at least one immunogen (e.g., DNA vaccine) and a combination of cytokine adjuvants, including macrophage inflammatory protein-1 alpha (MIP-1α) and FMS-related tyrosine kinase 3 ligand (Flt3L), or alternatively, macrophage inflammatory protein 3 alpha (MIP-3α) and Flt3L. This particular combination of adjuvants resulted in the induction of a vaccine-elicited immune response, which was unexpectedly more potent than those elicited by either adjuvant alone or any other combinations of adjuvants tested. The invention also features a “prime-boost” strategy, in which the vaccine of the invention is followed by the administration of a live vector boost using an expression vector (e.g., an adenovirus, a lentivirus, or a poxvirus, each of which includes a nucleic acid sequence encoding one or more immunogens) to further enhance vaccine immunogenicity. Thus, novel and practical strategies to induce strong cellular responses are provided herein to improve the efficiency of vaccines for the control of pathogenic states both in adults and neonates.

In a first aspect, the invention features a method for enhancing the immune response to an immunogen in a mammal (e.g., a human) by providing to the mammal the following polypeptides: an immunogen, Flt-3L or a biologically active fragment thereof, and MIP-1α, MIP-3α, or biologically active fragments thereof. Optionally, at least one, two, or all of the above polypeptides are provided to the mammal as expression vectors. Flt3L, MIP-1α, or MIP-3α may be any polypeptide substantially identical to the naturally occurring polypeptide (e.g., from mouse, human, rat, or monkey). For example, Flt3L, MIP-1α, or MIP-3α may be provided to the mammal being treated as the full-length polypeptides. Exemplary Flt3L and MIP-1α polypeptides are found in PCT WO 01/09303 A2, hereby incorporated by reference.

If desired, two, three, or more than three immunogens may be provided to the mammal being treated. According to the invention, the immunogen, Flt3L, and either MIP-1α or MIP-3α are provided in a therapeutically effective amount to augment the T cell response (CD4+ T cell response, CD8+ T cell response, or both) in the mammal; preferably, such response is increased by at least 20%, 30%, 40%, 50%, 60%, 70%, 80%, 90%, 95%, 100%, or even more than 100% relative to an untreated control. Optionally, other adjuvants (such as GM-CSF) may also be administered to the mammal being treated.

In all of the foregoing aspects of this invention, a booster shot may be administered to the mammal. Desirably, a booster shot is administered within a year of immunizing the mammal and may include one or more immunogens. Optionally, the booster shot may also include adjuvants such as MIP-1α, Flt3L, MIP-3α, or a combination thereof (e.g., Flt3L in combination with either MIP-1α, MIP-3α, or both) in a therapeutically effective amount. The booster shot may be a recombinant vector (at least 0.2 μg provided), which includes a polynucleotide sequence operably linked to regulatory elements encoding one or more immunogens. The recombinant vector may be a live recombinant vector (at least 10⁵ pfu provided). Exemplary live recombinant vectors include for example an adenovirus, a lentivirus, or a poxvirus (e.g., modified vaccinia virus Ankara, or fowl pox). According to the methods featured in this invention, the booster shot results in at least a 2-fold increase in the T cell response (CD4+ T cell response, CD8+ T cell response, or both) in the mammal compared to a control mammal not provided with the booster shot.

The methods featured by the present invention may be used to treat or prevent microbial infections (e.g., bacterial, viral such as HIV, fungal, or parasitic), autoimmune diseases, tissue rejection, allergic reactions, cancer (e.g., melanoma, breast, pancreatic, colon, lung, glioma, hepatocellular, endometrial, gastric, intestinal, renal, prostate, thyroid, ovarian, testicular, liver, head and neck, colorectal, esophagus, stomach, eye, bladder, glioblastoma, or metastatic carcinoma). Optionally, a second therapeutic agent, or regimen may also be provided to the mammal during, or within a week before, or after enhancing the immune response of the mammal. According to this invention, the mammal may be provided one, two, or more than two immunogens, and the immunogen is substantially identical to an antigen present in cancer (e.g., Melan-A, tyrosinase, p97, β-HCG, GalNAc, MAGE-1, MAGE-2, MAGE-3, MAGE-4, MAGE-12, MART-1, MUC1, MUC2, MUC3, MUC4, MUC18, CEA, DDC, melanoma antigen gp75, Hker 8, high molecular weight melanoma antigen, K19, Tyr1 and Tyr2, members of the pMel 17 gene family, c-Met, PSA, PSM, α-fetoprotein, thyroperoxidase, gp1000, NY-ESO-1, telomerase, C25 colon carcinoma, or p53, but preferably not a variable region of an immunoglobulin expressed by a B cell lymphoma), allergic reaction, tissue rejection, autoimmune diseases, microbial infections such as HIV (e.g., gp160, p24 VLP, gp41, p31, p55, gp120, Tat, gag, pol, env, nef, rev, or VaxSyn). Methods of this invention are particularly useful to immunize a neonate in order to prevent viral transmission during breastfeeding.

In all foregoing aspects of the invention, the method may also be used to substantially reduce the dosage or volume of vaccine required to immunize the mammal. The immunogen, or immunogens, Flt3L, MIP-1α, MIP-3α, and the booster shot may be formulated for injection intradermally, intramuscularly, subcutaneously, or intravenously and all polypeptides may be provided in the same formulation. If these polypeptides are not provided within the same formulation, they may alternatively be provided by the same route of administration, but no more than 20 cm apart on the surface of the mammal. For example, Flt3L and MIP-1α may be provided as recombinant polypeptides (each at a dose of at least at 0.1 ug/kg). Alternatively, the various polypeptides of the invention (including one or more immunogens, MIP-1α, and Flt3L) may be provided to the mammal by means of expression vectors containing polynucleotide sequences operably linked to regulatory elements. Expression vectors according to the present invention can be viral (e.g., adenovirus, poxvirus, and lentivirus), bacterial, or a plasmid vector. If provided as a viral vector, at least 10⁵ pfu of live recombinant virus is provided, and at least 0.2 ug of a plasmid, or bacterial vector is provided.

According to this invention, Flt3L, MIP-1α, and MIP-3α are delivered to a mammal as components of a vaccine formulation, either as recombinant polypeptides, or alternatively, by means of expression vectors. Thus, each of Flt3L, MIP-1α, and MIP-3α refers to any protein or nucleic acid molecule expression product that is substantially identical to the naturally occurring Flt3L, MIP-1α, and MIP-3α, respectively, biologically active derivatives thereof, or fragments thereof which enhance and/or modulate the immune response of a mammal to a vaccine. Preferably, these adjuvants are of murine, human, or monkey origin. Exemplary MIP-1α sequences can be found in GENBANK accession number U72395, NM 011337, and NM 002983. Exemplary Flt3L sequences can be found in GENBANK accession numbers NP 038548, AAH19801, NP001450, NM 013520, NM 001459, or BC 019801). Exemplary MIP-3α can also be found in GENBANK and include accession numbers AAB61459 and BAC55967. Exemplary GM-CSF sequences can be found in GENBANK accession number M11220, M11734, or M10663. Desirably, MIP-1α, MIP-3α, Flt3L, and GM-CSF are substantially identical to any of the naturally occurring adjuvant or any of the biological fragments thereof that exhibit the same biological activity as the naturally-occurring adjuvant. For example, MIP-1α and Flt3L may be substantially identical to any one of the polypeptides found in PCT WO 01/09303 A2, hereby incorporated by reference.

The adjuvant activity of Flt3L, MIP-1α, and MIP-3α is measured by any standard method in the art, such as the ability to enhance T (CD4+ or CD8+) cell response. Preferably, such response is increased by at least 10%, 20%, 30%, 40%, 50%, 60%, 70%, 80%, 90%, 95%, 100%, or preferably more than 100%, as measured by any method known in the art, such as by any one of the following methods: ELISPOT assay, tetramer binding assay, or cytotoxicity assay; alternatively, adjuvant activity is measured by the ability to induce T cell proliferation responses by at least 10%, 20%, 30%, 40%, 50%, 60%, 70%, 80%, 90%, 95%, 100%, or preferably more than 100%, as measured by any standard techniques including thymidine incorporation assays. Adjuvant activity may also be measured by the ability to induce and enhance antibody responses by at least 10%, 20%, 30%, 40%, 50%, 60%, 70%, 80%, 90%, 100%, or preferably more than 100% as measured by any standard method, such as by ELISA or by a neutralizing antibody assay.

By “allergic reaction” is meant a state of hypersensitivity of the immune system induced by the exposure to a particular antigen (allergen) resulting in harmful immunologic reactions on subsequent exposures. Allergic reactions are usually used to refer to hypersensitivity to an environmental antigen (atopic allergy or contact dermatitis) or to drug allergy.

By “augmenting T cell response” is meant to increase the T cell response to a vaccine by increasing the proliferation, the activity, or both of CD4+ T cells, CD8+ T cells, or both. Preferably, T cell proliferation is increased by at least 10%, 20%, 30%, 40%, 50%, 60%, 70%, 80%, 90%, 95%, preferably 100%, or even more preferably more than 100% over baseline levels, as measured by any method known in the art, including, for example, thymidine incorporation. Alternatively, T cell activity is increased by at least 10%, 20%, 30%, 40%, 50%, 60%, 70%, 80%, 90%, 95%, 100%, or even more than 100% over baseline levels, as measured by any method known in the art. Exemplary methods include tetramer binding assays, cytotoxicity assays, or ELISPOT assays.

By “autoimmune disease” is meant any condition in which an individual's immune system starts reacting against his or her own tissues by producing a self-directed humoral response, a cellular response, or both. Autoimmune diseases that result from such an abnormal immune response include for example rheumatoid arthritis (RA), multiple sclerosis (MS), insulin dependent diabetes mellitus (IDDM), arthritis, psoriasis, Crohn's disease, ulcerative colitis, and lupus.

By “biologically active fragment” is meant any polypeptide having an amino acid sequence that is substantially identical to the sequence of the naturally occurring adjuvant polypeptide of the invention and sharing a common biological activity with this adjuvant. According to this invention, the biologically active fragment may therefore increase the T cell response (CD4+ T cell response, CD8+ T cell response, or both) by at least 10%, 20%, 30%, 40%, 50%, 60%, 70%, 80%, 90%, 100%, or more than 100% relative to an untreated control as measured by any method described herein. Exemplary biologically active fragments are described in detail in PCT WO 01/09303, hereby incorporated by reference and provided as Appendix A.

By “booster shot” is meant a second or later vaccine composition that is provided to the mammal after the primary vaccine to increase the immune response to the original vaccine antigen(s). The vaccine given as the booster dose may be a DNA vaccine or a recombinant vector vaccine. If desired, the booster shot may contain the same formulation as the primary vaccine and may also contain the same immunogen as the first vaccine shot. Optionally, this booster shot may also be provided with adjuvants (e.g., MIP-1α, MIP-3α, Flt3L, and GM-CSF). Administration of a booster shot, according to this invention, increases the T cell response by 10%, 20%, 30%, 40%, 50%, 60%, 70%, 80%, 90%, more than 90%, or most preferably 100%, over the T cell response in mammals that have received the initial first shot but not the prime booster shot. T cell response may be measured by any standard method known in the art such as T cell proliferation, ELISPOT assay, tetramer binding assay, or cytotoxicity assay.

By “cancer” is meant is meant any condition characterized by an uncontrolled and abnormal accumulation of cells due to increased proliferation rates or decreased apoptotic rates, for example. Cancer cells can spread locally or through the bloodstream and lymphatic system to other parts of the body. According to the present invention, cancers include without limitation melanoma, breast, pancreatic, colon, lung, glioma, hepatocellular, endometrial, gastric, intestinal, renal, prostate, thyroid, ovarian, testicular, liver, head and neck, colorectal, esophagus, stomach, eye, bladder, glioblastoma, and metastatic carcinoma.

By “enhancing the immune response” is meant modulating a mammal's immune response by generating a cellular response, a humoral response, or both to impart a desirable therapeutic response by the administration of a vaccine, which may contain an immunogen. When administered to a mammal, the vaccine modulates the mammal's immune response sufficiently to decrease the symptoms and the causes of symptoms, or alternatively, eliminates or reduces causes of symptoms by increasing desirable immune response. According to this invention, the immune response of a mammal can be assessed according to their T cell response or antibody production. T cell response may be measured by any standard method known in the art, such as T cell proliferation, ELISPOT assay, tetramer binding assay, or cytotoxicity assay. Preferably, T cell response is increased by at least 10%, 20%, 30%, 40%, 50%, 60%, 70%, 80%, 90%, 95%, 100%, or preferably more than 100% above T cell response in the absence of vaccination. Desirably, immunogen-specific antibodies are increased by at least 10%, 20%, 30%, 40%, 50%, 60%, 70%, 80%, 90%, 95%, 100%, or preferably more than 100% above baseline values as measured by any standard technique such as by ELISA or by antibody neutralizing assay.

By “immunogen” is meant an antigen, or a peptide encoded by a vector, which augments the immune response according to the present invention. The target immunogen is therefore an immunogenic peptide or antigen, which is substantially identical to a naturally occurring antigen involved in pathological states. Such exemplary naturally occurring antigens include for example allergens, or any antigen associated with microbial infections, cancer, autoimmune disease, or transplantation rejection. Immunogens elicit an immune response directed against the target antigen, which will protect and/or treat the mammal against the specific infection or disease, with which the immunogen is associated.

By “providing” is meant administering to a mammal a composition (containing polypeptides (e.g., an immunogen, MIP-1α, MIP-3α, and Flt3L), nucleic acids (encoding, for example, an immunogen, MIP-1α, MIP-3α, and Flt3L), or mixtures thereof to enhance the immune response of the vaccinated mammal against a specific immunogen. According to this invention, vaccines include for example, a subunit vaccine, a killed vaccine, a live attenuated vaccine, a cell vaccine, a recombinant vaccine, or a nucleic acid (e.g., DNA) vaccine.

By “substantially identical” is meant a polypeptide or nucleic acid exhibiting at least 75%, but preferably 85%, more preferably 90%, more preferably 95%, even more preferably 99% identity, or most preferably 100% sequence identity to a reference amino acid or nucleic acid sequence. For polypeptides, the length of comparison sequences will generally be at least 20 amino acids, preferably at least 30 amino acids, more preferably at least 40 amino acids, and most preferably 50 amino acids. For nucleic acids, the length of comparison sequences will generally be at least 60 nucleotides, preferably at least 90 nucleotides, and more preferably at least 120 nucleotides.

By “substantially reducing the dosage of vaccine” is meant decreasing the total amount of vaccine encoding the immunogen to be provided to a mammal by the co-administration of adjuvants, such as MIP-1α, MIP-3α, Flt3L, and GM-CSF, while retaining the ability to augment T cell response in the vaccinated mammal. According to the present invention, the amount of vaccine is decreased by at least 2 fold, preferably by 4 fold, and most preferably by more than 4-fold below standard amount of vaccine administered without significantly affecting its biological activity.

By “tissue rejection” is meant the immune rejection and destruction of a graft (e.g., organ, tissue, or cell) following the recognition of the grafted material as foreign material by the host, and subsequent induction of an immune response directed to the graft.

By “vector” is meant a DNA molecule, usually a plasmid, a bacterial, or a viral vector, into which fragments of DNA may be inserted or cloned. A vector will contain one or more unique restriction sites, and may be capable of autonomous replication in a defined host or vehicle organism such that the cloned sequence is reproducible. A vector contains a promoter operably linked to a gene or coding region such that, upon transfection into a recipient cell, an RNA and protein are expressed. According to this invention, a bacterial, a viral, or a plasmid vector is a gene construct that contains the necessary regulatory elements operably linked to a coding sequence that encodes an immunogen, MIP-1α, MIP-3α, Flt3L, or a combination thereof, such that when present in the cell of a mammal, the coding sequence will be expressed. A vector according to this invention can be delivered topically (e.g., ointment, or patch), orally, or by injection (e.g., intramuscularly, intravenously, sub-cutaneously, or intraperitoneally).

The methods disclosed by the current invention may be used to markedly increase the immunogenicity and efficacy of virtually any vaccine and can therefore be used to immunize mammals against numerous pathological states, such as microbial infections (e.g., viral, bacterial, fungal, or parasitic), allergic reactions, cancer, autoimmune diseases, and transplantation rejection. Furthermore, the methods featured by this invention are also useful to substantially augment the immune response of a mammal prior to, during, or following treatment with a second therapeutic regimen. The co-delivery of MIP-1α and Flt3L or MIP-3α and Flt3L with a vaccine and the resultant synergistic adjuvant effect we have discovered is surprising and results in an immune response which is unexpectedly more potent, durable, versatile, and practical than any previously described cytokine adjuvant strategy. In addition to the induction of a robust cellular response involving both CD8+ and CD4+ cells, the immunogenicity of our vaccine formulation, can be further enhanced by a recombinant vector boost. An additional advantage of the present invention is that the greatly enhanced immune response allows a substantial reduction in the dosage and volume of a vaccine composition required to elicit a protective response. The vaccine formulations we provide allow the immunogen to be delivered in a reduced-dosage and/or reduced-volume injection. This provides advantages at the level of the patient, product development, and large-scale clinical use.

### BRIEF DESCRIPTION OF THE DRAWINGS

FIGS. 1A-1E are photographs showing the histopathology of injection sites. Balb/c mice (n=4/group) were immunized i.m. with (A) saline, (B) gp120 DNA vaccine alone, or gp120 DNA vaccine with (C) plasmid Flt3L, (D) plasmid MIP-1α, or (E) both plasmid Flt3L and plasmid MIP-1α. 50 μg of each plasmid was injected with sufficient sham plasmid to keep the total DNA dose per mouse constant. 5 μm muscle sections were stained with hematoxylin and eosin B (H&E) on day 7 following immunization (20× magnification).

FIGS. 2A-2G are photographs of immunohistochemical preparations of injection sites. 5 μm muscle sections from the vaccinated mice described in FIG. 1 were stained with mAbs specific for murine (A) CD3, (B) CD11b, (C) S100, (D) CD83, (E) MHC class II, (F) CD80, and (G) an isotype control (20× magnification).

FIGS. 3A-3D are bar graphs analyzing the DCs extracted from injected muscles. Balb/c mice were immunized as described in FIG. 1. On day 7 following immunization, muscles were excised, homogenized, and digested with collagenase and trypsin (n=8/group). Cell suspensions were analyzed by 4-color flow cytometry, and DCs were defined as gated CD3⁻CD19⁻ classII⁺CD11c⁺ cells. (A) Mean total number of extracted cells and (B) mean total number of extracted DCs and CD80^{hi} DCs per muscle are shown. (C) Percentage of total extracted cells that were DCs and (D) percentage of DCs that were CD80^{hi} are also shown. In all samples, <5% of the cells were CD3⁺ or CD19⁺ lymphocytes.

FIG. 4 is a graph showing T cell response over time, as measured by tetramer binding assay, of mice immunized with an empty DNA vaccine vector or a DNA vaccine encoding HIV-1 gp120, alone, or in combination with: MIP-1α; MIP-1α, and GM-CSF; Flt3; MIP-1α, and Flt3L; and MIP-1α, Flt3L, and GM-CSF.

FIGS. 5A-5C are graphs showing the immunogenicity of MIP-1α/Flt3L-augmented DNA vaccines. Balb/c mice (n=8/group) were immunized with sham plasmid, gp120 DNA vaccine alone, or gp120 DNA vaccine with plasmid Flt3L, plasmid MIP-1α, or both plasmid Flt3L and plasmid MIP-1α. 50 μg of each plasmid was injected with sufficient sham plasmid to keep the total DNA dose per mouse constant. Vaccine-elicited immune responses were assessed by (A) D^{d}/P18 tetramer binding to CD8+ T lymphocytes, (13) Env pooled peptide and P18 epitope peptide-specific ELISPOT assays, and (C) gp120-specific ELISAs.

FIG. 6 is a graph showing T cell response, as measured by a tetramer binding assay, in mice immunized with a DNA vaccine encoding HIV-1 gp120, alone or in combination with: Flt3L, GM-CSF, and MIP-1α or Flt3L, GM-CSF, and MIP-3α. The figure also shows the T cell response of mice injected with the DNA vaccine in the left leg and the combination of Flt3L, GM-CSF, and MIP-1α in the right leg.

FIGS. 7A and 7B are graphs showing the generalizability of MIP-1α/Flt3L-augmented DNA vaccines. (A) Balb/c mice or C57/BL6 mice were immunized, respectively, with 50 μg HIV Env gp120 DNA vaccine or 50 μg SIV Gag DNA vaccine, each with or without plasmid MIP-1α and plasmid Flt3L. ELISPOT assays were performed using pooled Env peptides and the P18 epitope peptide for the Env-vaccinated mice, or pooled Gag peptides and the AL11 epitope peptide for the Gag-vaccinated mice. (B) ELISPOT assays were performed using splenocytes from Env-vaccinated Balb/c mice depleted of CD4⁺ or CD8⁺ T lymphocytes.

FIGS. 8A and 8B are graphs showing secondary responses following MIP-1α/Flt3L-augmented DNA vaccine priming and DNA vaccine boosting. Balb/c mice (n=4/group) were primed with 50 μg gp120 DNA vaccine with or without (A) plasmid MIP-1α and plasmid Flt3L or (B) plasmid MIP-1α and plasmid CD40L. At week 6 following immunization, all mice were boosted with 50 μg gp120 DNA vaccine. Vaccine-elicited cellular immune responses were assessed by D^{d}/P18 tetramer binding to CD8⁺ T lymphocytes following the boost.

FIG. 9 is a graph showing the augmentation of T cell response following a second booster shot with recombinant adenovirus type 5 (rAd5) encoding HIV-1 gp120, as measured by tetramer binding assay. Mice were immunized with an empty DNA vaccine vector or a DNA vaccine encoding HIV-1 gp120, alone, or in combination with: MIP-1α; MIP-1α and GM-CSF; Flt3L; MIP-1α and Flt3L; and MIP-1α, Flt3L, and GM-CSF.

FIG. 10 is a graph showing T cell response over time, as measured by tetramer binding assay, of mice immunized with an empty DNA vaccine vector or a DNA vaccine encoding HIV-1 gp120, alone, or in combination with: Flt3L; Flt3L and GM-CSF; MIP-1α and Flt3L; and MIP-1α.

FIG. 11 is a graph showing the augmentation of T cell response following a second booster shot using a recombinant adenovirus type 5 (rAd5) vector, as measured by tetramer binding assay. Mice were immunized with an empty DNA vaccine vector or a DNA vaccine encoding HIV-1 gp120, alone, or in combination with: CD40L; CD40L and GM-CSF; MIP-1α and CD40L; and MIP-1α.

FIGS. 12A-12D are graphs showing the results of mechanistic studies of plasmid MIP-1α and plasmid Flt3L. (A) Balb/c mice were immunized i.m. with sham plasmid, gp120 DNA vaccine alone, gp120 DNA vaccine mixed with plasmid MIP-1α and plasmid Flt3L and delivered equally in both legs, or gp120 DNA vaccine in the left leg and plasmid MIP-1α and plasmid Flt3L in the right leg. (B) Mice were immunized with the gp120 DNA vaccine with or without plasmid MIP-1α and plasmid Flt3L and received daily i.v.+i.p. injections of saline, 1 μg human MIP-1α protein, or 1 μg murine MIP-1α protein for 3 days. (C) Mice were immunized with the gp120 DNA vaccine with or without plasmid MIP-1α and plasmid Flt3L at doses of 50 μg, 5 μg, or 0.5 μg of each plasmid in 50 μl injection volumes. (D) Mice were immunized with the gp120 DNA vaccine with or without plasmid MIP-1α and plasmid Flt3L at doses of 50 μg of each plasmid in 50 μl or 15 μl injection volumes. Vaccine-elicited cellular immune responses were assessed by D^{d}/P18 tetramer binding to CD8⁺ T lymphocytes on day 10 following immunization.

FIGS. 13A and 13B are graphs showing the immune response to recombinant vaccinia virus challenge. Balb/c mice (n=4/group) were immunized i.m. with sham plasmid, gp120 DNA vaccine, or gp120 DNA vaccine with plasmid Flt3L and plasmid MIP-1α. At week 12 following immunization, mice were challenged i.p. with 10⁷ pfu recombinant vaccinia virus expressing HIV-1 Env IIIB. (A) Anamnestic immune responses were assessed by D^{d}/P18 tetramer binding to CD8+ T lymphocytes following challenge. (B) Vaccinia virus titers (pfu) were assessed in ovaries harvested on day 7 following challenge.

### DETAILED DESCRIPTION

In general, the present invention features methods to substantially increase the immunogenicity of a vaccine, preferably a DNA vaccine, and involves the administration of a specific combination of cytokine adjuvants.

Given that a major limitation of DNA vaccines is their limited immunogenicity in primates, one strategy to augment the immune response to antigens encoded by such vaccines involves the administration of cytokine adjuvants. Cytokine adjuvants can alter the type and intensity of the vaccine-mediated T cell response by increasing the migration and recruitment of macrophages and dendritic cells to the site of injection, for example. In turn, dendritic cells and macrophages play a critical role in the T cell response as they specialize in the uptake of antigen and their presentation to T cells.

Dendritic cells (DCs), in particular, are critical for priming adaptive immune responses to foreign antigens. DCs are antigen-presenting cells that play a central role in priming immune responses to foreign antigens. Following activation by lipopolysaccharide, cytokines, or other stimuli, immature DCs upregulate expression of MHC and costimulatory molecules and develop into mature DCs that prime T lymphocytes with extraordinary efficiency. This process initiates immune responses against invading pathogens effectively.

Although cytokine adjuvants can alter the type and intensity of the vaccine-mediated T cell response, their effects are typically weak and limited. This invention provides a vaccine regimen, which involves administering to a mammal a composition that includes at least one immunogen (e.g., which may be specific to a pathological state), Flt3L, and either MIP-1α or MIP-3α, within the same local area. This invention is based on our discovery that the immune responses induced as a result of the co-administration of Flt3L with either MIP-1α or MIP-3α with a vaccine are surprisingly superior to the immune responses generated by any other combinations of adjuvants tested (e.g., CD40L alone; CD40L and GM-CSF; MIP-1α and CD40L; and MIP-1α alone). According to this invention one adjuvant recruits antigen-presenting cells (APCs) to the site of inoculation while the other induces the activation, proliferation, and maturation of these cells resulting in a potent and durable augmentation of immune responses elicited by a vaccine. According to the present invention, the immunogen, Flt3L and either one of MIP-1α or MIP-3α may be administered, together or separately, as recombinant polypeptides, or more preferably by way of nucleic acids, which encode the proteins. If desired, other adjuvants such as GM-CSF may also be administered to the mammal.

Desirably, the immunogenicity of this vaccine strategy is further augmented by the administration of a second booster shot. Since the adjuvant combinations of the invention induce a strong, rapid, and durable cellular immune response, particularly when given with a booster shot, vaccines provided according to this invention may be used to prevent viral infections (e.g. vertical transmission of HIV through breastfeeding or horizontal transmission of HIV through body fluid or sexual contact). Because of the general applicability of the methods disclosed, the present invention may be used to immunize a mammal to treat or prevent against microbial infections, including but not limited to HIV; hyperproliferative diseases such as cancer and psoriasis; autoimmune diseases; allergic reactions; and tissue rejection. Optionally, this invention is also useful to immunize a mammal prior to treatment, during treatment, or following treatment with a second therapeutic regimen against those same conditions. In addition to humans, the methods of the present invention may be used to immunize other mammals including, for example, a monkey, ape, cow, sheep, sheep, goat, buffalo, antelope, horse, mule, donkey, deer, elk, caribou, buffalo, camel, llama, alpaca, rabbit, pig, mouse, rat, guinea pig, hamster, dog, or cat.

Plasmids

The expression vector of the invention may be a DNA or RNA molecule encoding at least one immunogen, Flt3L, MIP-1α, or MIP-3α, or a combination thereof. For example, the immunogen and Flt3L may be administered as plasmid DNA molecules, while MIP-1α is administered as a recombinant polypeptide. As another example, the immunogen, Flt3L, or MIP-3α are all administered as DNA plasmids. In cases in which an additional adjuvant (e.g., GM-CSF) is administered to the mammal, this adjuvant may be a nucleic acid molecule or a recombinant polypeptide.

Sequences that encode the immunogen may occur on a separate or the same nucleic acid molecule as the nucleic acid molecule that contain the sequences that encode Flt3L, MIP-1α, or MIP-3α. The DNA vaccine can include, for example, a plasmid or a viral vector, such as an adenovirus, poxvirus, retrovirus, or lentivirus. The vectors encoding the immunogen, Flt3L, MIP-1α, MIP-3α, or a combination thereof, are linked to regulatory elements necessary for expression within the cells of a vaccinated mammal. Regulatory elements for DNA expression include initiation and termination signals such as a promoter and polyadenylation signal, capable of directing the expression of the immunogen, Flt3L, MIP-1α, MIP-3α, or combination thereof in the cells of the vaccinated mammal. Other exemplary regulatory elements, such as a Kozak region for example, may also be included in the genetic construct.

Immunogens

The immunogen of the vaccine may be delivered directly (e.g. as a peptide or several peptides), or more preferably by means of a nucleic acid sequence encoding the immunogen, which is included in a delivery vector. For the vaccine regimen disclosed in this invention, vectors encoding immunogens contain at least one epitope identical or substantially identical to an epitope associated with the pathological state. At least one immunogen, two, three, preferably more than three, can be included in one vector, and desirably at least one, two, three, or more immunogens are formulated in the vaccine regimen. The immunogen may be any molecular moiety against which an increase or decrease in immune response is sought. This includes immunogens derived from organisms known to cause diseases in mammals such as bacteria, viruses, parasites and fungi; antigens expressed by tumors, or abnormal host cells in autoimmune diseases, and allergens. Different combinations of immunogens may be used that show optimal function with different ethnic groups, sex, geographic distributions, and stage of diseases. Preferably, the injection of the vector encoding the immunogen into a mammal increases the T cell response (CD4+ T cell response, CD8+ T cell response, or both) by at least 10%, 20%, 30%, 40%, 50%, 60%, 70%, 80%, 90%, 100%, or more than 100% above baseline levels as measured by any standard method known in the art, including for example, T cell proliferation, ELISPOT assay, tetramer binding assay, or cytotoxicity assay. Alternatively, the vector may also induce a humoral response, increasing the production of an immunogen specific antibody by at least 10%, 20%, 30%, 40%, 50%, 60%, 70%, 80%, 90%, more than 90%, or more preferably 100% above baseline levels, as measured by any standard techniques such as an ELISA or neutralizing antibody assay.

Adjuvants: Combination of Flt3L and MIP-1α or Flt3L and MIP-3α

The present invention discloses the combination of Flt3L with MIP-1α or MIP-3α as potent vaccine adjuvants. If desired, such combinations may also include other adjuvants, such as GM-CSF. When co-delivered with a DNA valccine, Flt3L and MIP-1α, or alternatively Flt3L and MIP-3α, synergize to induce a durable and potent immune response, driven in part by T cells. These results are surprising as the immunogenicity induced by these vaccines was far more superior than any other combinations tested, including CD40L alone; CD40L and GM-CSF; MIP-1α and CD40L; and MIP-1α alone.

Flt3L and either one of MIP-1α or MIP-3α may be delivered either alone, together, or in combination with the immunogen, either as polypeptides or by means of a vector (e.g., plasmid or viral vector). Viral vectors, according to the present invention, include without limitation viral vector including adenovirus, poxvirus, retrovirus, or lentivirus. Preferably, the Flt3L, MIP-1α, or MIP-3α polypeptides of the invention have an amino acid sequence substantially identical to the natural product or a recombinant protein derived from the natural product; the recombinant polypeptide may thus include modifications that changes its pharmacokinetic properties while keeping its original chemattractant property. Alternatively, the recombinant polypeptides may be identical to the naturally occurring compound. Although the adjuvants of the invention may be of any origin, these adjuvants are preferably murine, human, or monkey polypeptides. Exemplary MIP-1α sequences may be found in GENBANK accession number U72395, NM 011337, and NM 002983. Exemplary Flt3L sequences can be found in GENBANK accession numbers NP 038548, AAH19801, NP001450, NM 013520, NM 001459, or BC 019801. Exemplary MIP-3α can also be found in GENBANK and include accession numbers AAB61459 and BAC55967. Desirably, the MIP-1α, MIP-3α, and Flt3L polypeptides of the invention are substantially identical to any one of the corresponding and naturally-occurring adjuvant or fragment thereof that displays the same biological activity as the naturally-occurring adjuvant. Even more desirably, these polypeptides exhibit at least 10%, 20%, 30%, 40%, 50%, 60%, 70%, 80%, 90%, 95%, 100%, or more than 100% of the biological activity of the corresponding naturally occurring polypeptides. For example, MIP-1α and Flt3L may be substantially identical to any one of the polypeptides and fragments described in PCT WO 01/09303 A2, hereby incorporated by reference.

Microbial Infections

The present invention is useful to immunize a mammal against a wide range of pathogens, including for example viruses (e.g., HI), prokaryotes, and pathogenic eukaryotic organisms such as unicellular pathogenic organisms and multicellular parasites. This invention is particularly useful to immunize against pathogens, which infect cells and are not encapsulated, such as viruses. Of course, to produce a vaccine regimen that protects or treats pathogenic infections, the immunogen encoded by the vaccine must induce an immune response in the mammal, and is substantially identical or identical to an antigen characteristic of the pathogen. Desirably, the genetic construct used in the vaccine includes a DNA sequence, which encodes at least one, preferably two or more immunogens. For example, several viral genes may be included in a single vector to provide multiple targets. As a specific example, a genetic construct encodes for a protein, or a peptide substantially identical to env and the rev gene, or alternatively a peptide substantially identical to the gag, pol and env gene may also be used to immunize a mammal to HIV-1 infection. Optionally, a vaccine according to the methods of the present invention may also be provided to a mammal before, during or after treatment with a second anti-microbial therapeutic regimen. For example, a vaccine regimen comprising a DNA vaccine encoding gp120, Flt3L, and MIP-1α followed by a boost shot, may be provided to a patient with HIV during or before the administration of an anti-viral regimen. Such an anti-viral regimen may include for example, a highly active anti-retroviral therapy (HAART), which is a therapy composed of multiple anti-HIV drugs.

Hyperproliferative Diseases

The present invention also provides methods for treating or preventing hyperproliferative diseases by eliciting a protective immune response against hyperproliferating cells. An immune response is typically generated against a target antigen produced by such cells. Examples of such diseases include, for example, cancer and psoriasis. Optionally, a vaccine according to the methods of the present invention may also be provided to a mammal before, during, or after treatment with a second therapeutic regimen against such hyperproliferative diseases. For example, this vaccine can be provided to a cancer patient before or after anti-neoplastic therapy (e.g., radiotherapy, or chemotherapy) to further increase the anti-cancer efficacy in the mammal.

To immunize or treat a mammal against such hyperproliferative diseases, a DNA vaccine regimen containing a construct that includes a nucleotide sequence, encoding a protein associated with a hyperproliferative disease, is administered to a mammal. In order for the hyperproliferative-associated protein to be an effective immunogenic target, a potential immunogen is produced exclusively or at higher levels in hyperproliferative cells as compared to normal cells. In some cases, a hyperproliferative-associated protein is the product of a mutation of a gene that encodes a protein. Such a protein is nearly identical to the normal protein except it has a slightly different amino acid sequence, which results in a different epitope not found on the normal protein. Examples of such proteins are encoded by oncogenes such as myb, myc, fyn and the translocation gene bcr/abl, ras, src, p53, neu, trk, and EGFR. In addition to oncogene products as candidate immunogens, immunogens may also include variable regions of antibodies made by B cell lymphomas and variable regions of T cell receptors of T cell lymphomas, which are also used for autoimmune diseases. Other tumor-associated proteins that can be used as target proteins include for example proteins that are selectively overexpressed in tumor cells, or tumor associated cells. Preferably, the target antigen is not a variable region of an immunoglobulin expressed by the B-cell lymphoma.

Both primary and metastatic cancers can be treated in accordance with the invention. Cancers which can be treated include without limitation melanoma, breast, pancreatic, colon, lung, glioma, hepatocellular, endometrial, gastric, intestinal, renal, prostate, thyroid, ovarian, testicular, liver, head and neck, colorectal, esophagus, stomach, eye, bladder, glioblastoma, and metastatic carcinoma. In particular, the present invention may be used to prophylactically immunize an individual who is predisposed to develop a particular cancer. Using genetic screening and/or family health history, it is possible to predict the associated probability and risk for reoccurrence of the cancer. Individuals who have already developed cancer and who have been treated with anti-cancer therapy or are otherwise in remission, are particularly susceptible to relapse and reoccurrence. As part of the treatment regimen, such individuals may be immunized against the cancer that they have been diagnosed as having had in order to prevent reoccurrence.

Immune Disorders

The present invention also provides methods of preventing and treating individuals against autoimmune diseases and disorders by conferring a broad based protective immune response against targets that are associated with autoimmunity, including cell receptors and cells which produce “self”-directed antibodies.

T-cell mediated autoimmune diseases amenable to prevention and/or treatment include without limitation Rheumatoid arthritis (RA), multiple sclerosis (MS), insulin dependent diabetes mellitus (IDDM), arthritis, psoriasis, Crohn's disease, and ulcerative colitis. Each of these diseases is characterized by T cell receptors that bind to endogenous antigens and initiate the inflammatory cascade associated with autoimmune diseases. Vaccination against the variable region of the T cells may elicit an immune response to eliminate such autoreactive T cells. Tissue rejection during transplantation and allergic reactions are also amenable to the methods disclosed in the present invention, in cases in which the T-cell mediated immune response may further necessitate immunomodulation.

Common structural features among the variable regions of both TCRs and antibodies are well known in the art. The DNA sequence encoding a particular TCR or antibody can generally be found following well known methods such as those described in Kabat et al. 2987 Sequence of Proteins of Immunological Interest U.S. Department of Health and Human Services, Bethesda Md., which is incorporated herein as a reference. In addition, a general method for cloning functional variable regions from antibodies can be found in Chaudhary et al., 1990 Proc. Natl. Acad. Sci. USA 87:1066, which is incorporated herein as a reference.

Formulation and Routes of Administration

According to the present invention, the immunogen, Flt3L, and either one of MIP-1α or MIP-3α are delivered in the mammal in a pharmaceutically acceptable carrier, alone or using any combination thereof. Desirably, the immunogen, Flt3L, and either one of MIP-1α or MIP-3α are administered in a single pharmaceutical composition consisting of an effective amount of Flt3L, and either one of MIP-1α or MIP-3α with an immunogen in a pharmaceutically acceptable carrier. According to this invention, the immunogen, Flt3L, either one of MIP-1α or MIP-3α, or a combination thereof may or may not be provided with the booster shot. Alternatively, the immunogen, Flt3L, either one of MIP-1α or MIP-3α, or a combination thereof are administered in separate formulations within at least 1, 2, 4, 6, 10, 12, 18, 24 hours, or more than 24 hours apart. The immunogen, Flt3L, and either one of MIP-1α or MIP-3α may also be administered by different routes of administration. Preferably, the immunogen, Flt3L, and either one of MIP-1α or MIP-3α are delivered within at least 20, 10, 5, 1 cm or less than 1 cm on the surface of the skin but most preferably at the same site and in the same formulation. Optionally, Flt3L, MIP-1α, MIP-3α, or a combination thereof can be delivered within a half hour, 1 hour, 2 hours, 4 hours, 6 hours, 12 hours, 24 hours or more than 24 hours before or after the administration of the immunogen.

These reagents may be combined and used with additional active or inert ingredients, e.g., in conventional pharmaceutically acceptable carriers. A pharmaceutical carrier can be any compatible, non-toxic substance suitable for the administration of the compositions of the present invention to a mammal. Pharmaceutically acceptable carriers include for example water, saline, buffers and other compounds described for example in the Merck index Merck & co. Rahway, N.J. Slow release formulation or a slow release apparatus may be also be used for continuous administration.

Concentrations of the immunogen, Flt3L, and either one of MIP-1α or MIP-3α necessary for effective vaccination will depend upon different factors, including means of administration, target site, physiological state of the mammal, and other medication administered. Thus treatment dosages may be titrated to optimize safety and efficacy. Typically, dosage ranges for the immunogen, the recombinant Flt3L, MIP-1α, or MIP-3α polypeptides are lower than 1 mM concentrations, typically less than about 10 uM concentrations, usually less than, about 100 nM, typically less than about 10 pM, and most preferably less than about 1 femtomolar or fM with an appropriate carrier. Treatment may be initiated with smaller dosages, which are less than the optimum dose of the compound. Thereafter, the dosage is increased by small increments until the optimum effect under the circumstance is reached. Determination of the proper dosage and administration regime for a particular situation is within the skill of the art.

According to the present invention, administration of plasmids encoding the immunogen, Flt3L, MIP-1α, MIP-3α, or any combination thereof, into a mammal comprise about 1 nanogram to about 5000 micrograms of DNA. Desirably, compositions comprise about 5 nanograms to 1000 micrograms of DNA, 10 nanograms to 800 micrograms of DNA, 0.1 micrograms to 500 micrograms of DNA, 1 microgram to 350 micrograms of DNA, 25 micrograms to 250 micrograms of DNA, or 100 micrograms to 200 micrograms of DNA. Alternatively, administration of recombinant adenoviral vectors (e.g., rAd5) encoding the immunogen, Flt3L, MIP-1α, MIP-3α, or any combination thereof, into a mammal may be administered at a concentration of at least 10⁵, 10⁶, 10⁷, 10⁸, 10⁹, 10¹⁰, or 10¹¹ plaque forming unit (pfu). The pharmaceutical compositions according to the present inventions are formulated according to the mode of administration to be used. In cases where pharmaceutical compositions are injectable pharmaceutical compositions, they are sterile, pyrogen-free and particulate free. An isotonic formulation is preferably used. Generally, additives for isotonicity can include for example sodium chloride, dextrose, mannitol, sorbitol and lactose. Stabilizers may also be used and include for example gelatin and albumin. A vasoconstriction agent can also be added in the formulation.

Overall, the composition consisting of at least one immunogen, Flt3L, MIP-1α, MIP-3α, or a combination thereof can be provided by injection (e.g., intrasmuscular, intranasal, intraperitoneal, intradermal, subcutaneous, intravenous, intraarterial, or intraoccular), as well as by oral, topical (e.g., ointment, or patch), or transdermal administration. Alternatively, these compositions may be provided by inhalation, or by suppository. Compositions according to the invention may also be provided to mucosal tissue, by lavage to vaginal, rectal, urethral, buccal, and sublingual tissue for example.

The preferred biologically active dose of Flt3L and either one of MIP-1α or MIP-3α to be delivered with the inmunogen within the practice of the present invention is a dosing combination that will induce the maximum in a CD4+ and CD8+ T cell response, as measured by tetramer binding assay, ELISPOT assay, cytotoxicity assays, or lymphoproliferation assays. Preferably, such increase is at least 20%, 30%, 40%, 50%, 60%, 70%, 80%, 90%, 95%, 99%, 100%, or even more than 100% over the level of a control vaccine which has not been administered with any adjuvants. Presumably, the combination of Flt3L and either one of MIP-1α or MIP-3α increases the migration and proliferation of antigen presenting cells at the site of injection of the antigen.

Booster Shots

The present invention also features methods of augmenting a potent immune response using a prime-boost strategy as an effective way to further enhance the immunogenicity of a DNA vaccine administered with Flt3L and either one of MIP-1α or MIP-3α. By itself, the initial DNA vaccine with Flt3L and either one of MIP-1α or MIP-3α augments robust vaccine-mediated T cell responses and antibody responses. The prime-boost combination, however, can stimulate a much more potent and durable cellular immune response, including persistent killer CD8+ T cells, as well as antibodies that can neutralize the naturally occurring antigen. Thus, the immune system of a mammal is initially primed with a vaccine regimen consisting of a DNA vaccine (prime vaccine) encoding an immunogen and the combination of Flt3L and either one of MIP-1α or MIP-3α, such as a DNA vaccine genetically engineered to contain a synthetic HIV gene, and subsequently the immune responses generated by this prime vaccine can be further boosted with the same or a different vaccine, containing the same or different immunogen. Optionally, the second, boost vaccine may be provided with Flt3L, either one of MIP-1α or MIP-3α, or both. Preferably, the boost vaccine is administered within at least 12, 6, 5, 4, 3, 2, one month, or less than one month of the initial vaccine, and within at least 30, 25, 20, 15, 10, 5, 1, or less than 1 cm apart from the initial vaccine site. As an example, a DNA vaccine is engineered to carry a foreign HIV gene(s), such as a subunit of the gp120 gene, and is administered to a mammal in the arm, or leg along with a biologically active formulation of plasmid encoding Flt3L and MIP-1α. There, the vaccine directs cells to make the gp120 immunogen protein, which in turn, stimulates production of protective T cells. Within two to six months, the mammal receives a booster shot of a different vaccine consisting of an adenovirus vector encoding the same immunogen, with or without Flt3L and MIP-1α.

Examples of vaccines that can be used in prime booster shots include for example DNA vaccines, adenovirus vaccines, vaccinia virus, canarypox virus, Salmonella. Preferably, DNA priming is followed by the administration of a booster consisting of recombinant modified vaccinia Ankara (rMVA), or recombinant human adenovirus 5 (rAd5) encoding the immunogen used in the priming shot. Both of rMVA and rAd5 have a broad host range for human cells and stimulate the production of pro-inflammatory cytokines that can augment immune responses by producing higher expression levels of immunogens or by stimulating a pro-inflammatory response. Booster shots may encode the same immunogen as the vaccine of the prime vaccine, or can alternatively encode different immunogens. Preferably, the prime booster shot is administered intramuscularly, intravenously, intraperitoneally, or sub-cutaneously. The vaccine may also consist of Flt3L, MIP-1α, MIP-3α, or a combination thereof, as described above for the primary vaccine composition.

Prevention of Vertical Transmission

The invention also provides methods to fulfill the need for a pediatric vaccine for the immunization of neonates against viral infection for example. In the case of HIV infection for example, 75% of postnatal transmission occur within 6 months of age and consequently typical immunization regimens, which involve 3-6 immunizations over 6-10 months frame of time, are not optimal for pediatric prophylactic vaccination. Although DNA vaccines have previously been shown to illicit an immune response in neonates, much stronger and rapid immune responses are needed, especially since neonates generate a weaker immune response. The present invention therefore fulfills this need by disclosing a method for enhancing the immunogenicity of pediatric vaccines. According to the methods of this invention, a vaccine regimen is provided and consists of a vaccine encoding at least one immunogen, Flt3L, and either one of MIP-1α or MIP-3α. This regimen may induce a strong and rapid immune response in the neonate mammal to prevent or attenuate postnatal HIV-1 transmission. Immunization as described by the present invention may also provide a degree of protection against HIV-1 transmission later in life. Neonates may be provided with a vaccine regimen comprising 0.5, 5, 50, 500, or 5000 micrograms of an HIV DNA vaccine with Flt-3L and either one of MIP-1α or MIP-3α, preferably within 24 hours, 48 hours of birth, more than 48 hours, or 1, 2, 3, 4, 5, 8, 10 weeks of birth, and can be administered by injection (e.g., intramuscular, intravenous, sub-cutaneous, or intraperitoneally), by topical, or oral administration. As an example, the initial vaccine would consist of four plasmids: an env encoding DNA vaccine, a gag-pol-nef encoding DNA vaccine, a plasmid encoding GM-CSF and a plasmid MIP-1α. Each plasmid will be administered at a weight-adjusted dose of 1 mg/kg (maximum dose of 5 mg each). Neonates can then be boosted once intramuscularly with rAd5-env and rAd5 encoding gag-pol-nef each at weight adjusted dose of 2×10⁹ pfu/kg (maximum of 10¹⁰ pfu each). The immunogenicity of each of these two injections can be determined by assessing vaccine-elicited immune response weekly for 16 weeks following primary immunization using methods described below.

Assessment of Immunogenicity

Heparin anticoagulated blood may be obtained at different time points following immunization with the vaccine regimen described in the present invention and vaccine-elicited T cell responses may be measured by pooled peptide interferon-gamma ELISPOT assay, tetramer binding assays, cytotoxicity assays, intracellular cytokine assays and lymphoproliferation. Humoral responses generated by the immunization with the vaccine regimen may be measured by ELISA or neutralizing antibody assays.

Assessment of T Cell Response

Following immunization of the mammal with the methods disclosed by the present invention, T cell response may be assessed by a number of methods. The overall T cell response may be assessed, for example, by measuring IFN-γ production by immunogen-specific T cells in an ELISPOT assay. In this assay, antigen-presenting cells (APC) are immobilized on the plastic surface of a microtiter well, and T cells are added at various T cell: APC ratios. Binding of APCs by antigen-specific effector cells triggers the production of cytokines, such as IFN-γ, by the T cells. Cells can be stained to detect the presence of intracellular IFN-γ and the number of positively staining foci (spots) counted under a microscope correlates with T cell response.

A second method for quantifying the number of circulating antigen-specific CD8+ T cells is the tetramer-binding assay. In this assay, a specific epitope is bound to synthetic tetrameric forms of fluorescently labeled MHC Class I molecules. Since CD8+ T cells recognize antigen in the form of short peptides bound to Class I molecules, cells with the appropriate T cell receptor will bind to the labeled tetramers and can be quantified by flow cytometry. Although this method is less time-consuming than the ELISPOT assay, the tetramer assay measures only binding, not function. Not all cells that bind a particular antigen necessarily become activated.

Alternatively, T cell response may be quantified by assays measuring lymphoproliferation such as thymidine incorporation assays. Such methods are described for example, by Barouch et al. (Barouch et al., J. Immunol. 168: 562-568 (2002)), herein incorporated by reference.

The following examples are intended to illustrate the principle of the present invention and circumstances in which the immunogenicity of a vaccine is augmented by the combination of Flt3L and either MIP-1α or MIP-3α are indicated. The following examples are not intended to be limiting.

### EXAMPLE 1

### Plasmid MIP-1α and Plasmid Flt3L Recruit and Expand DCs at the Site of Inoculation

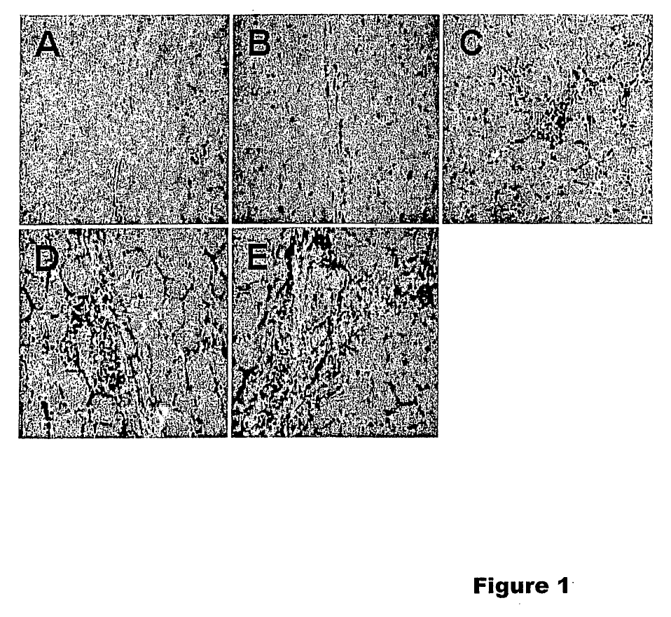

Studies were initiated to determine whether codelivering DC-specific chemotactic and growth factors with a plasmid DNA vaccine would lead to increased recruitment and expansion of DCs at the site of vaccine inoculation. We assessed the extent and nature of local cellular inflammatory infiltrates following intramuscular injection of plasmid DNA vaccines with or without plasmids expressing MIP-1 a and Flt3L. Groups of Balb/c mice (n=4/group) were immunized i.m. with sterile saline or 50 μg plasmid DNA vaccine expressing HIV-1 IIIB Env gp120 (Barouch et al., J. Immunol. 168:562-568 (2002)). Certain DNA vaccinated groups were coimmunized with 50 μg plasmid Flt3L, 50 μg plasmid MIP-1α, or both 50 μg plasmid MIP-1α and 50 μg plasmid Flt3L. Sufficient sham plasmid was included to keep the total dose of DNA per animal constant. The injected muscles were excised on day 7 following immunization, frozen immediately in OCT medium in a dry ice/methanol bath. Frozen muscles were cut into 5 μm thickness, air dried, and fixed for 10 min in 100% acetone. Fixed sections were stained with hematoxylin and eosin B (H&E) before dehydration, mounting, and examination for the presence and extent of cellular inflammatory infiltrates. As shown in FIG. 1, small infiltrates were observed following injection of the DNA vaccine alone. Coimmunization of plasmid Flt3L with the DNA vaccine resulted in slightly larger clusters of inflammatory cells; In contrast, large cellular infiltrates were recruited by plasmid MIP-1α or the combination of both plasmid MIP-1α and plasmid Flt3L. Quantitation of these inflammatory infiltrates demonstrated that coadministration of both of these plasmid cytokines resulted in >10-fold greater recruitment of inflammatory cells as compared with the DNA vaccine alone.

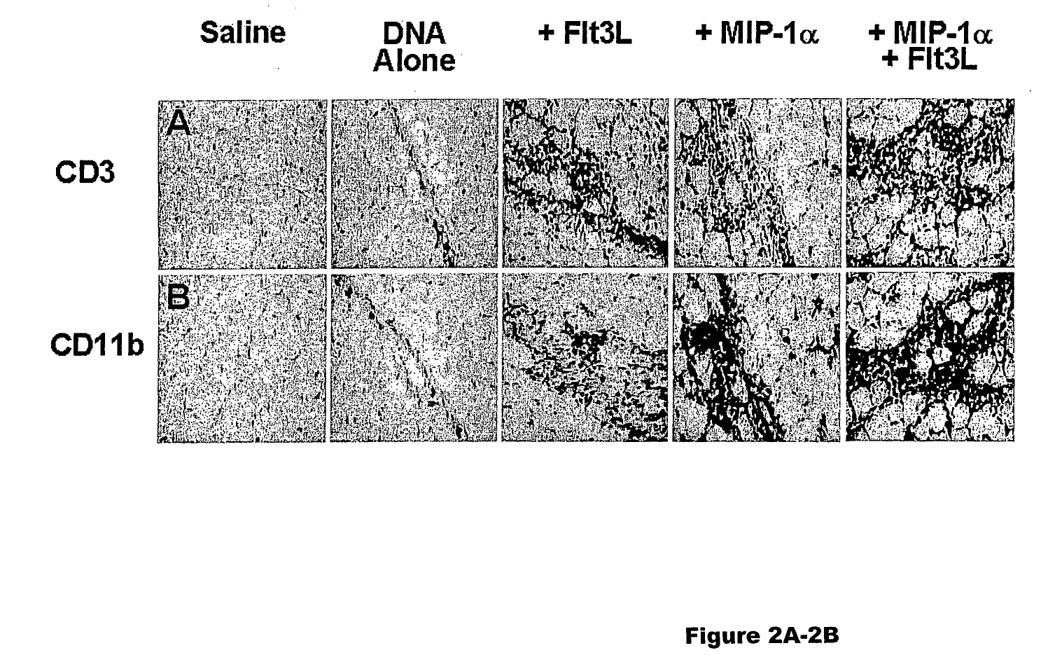

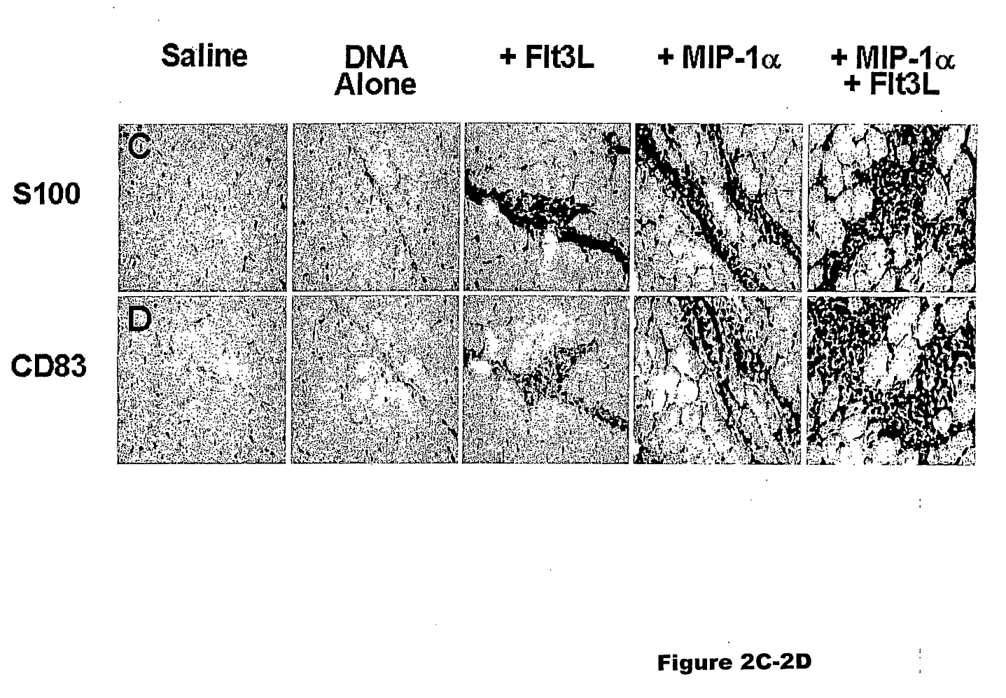

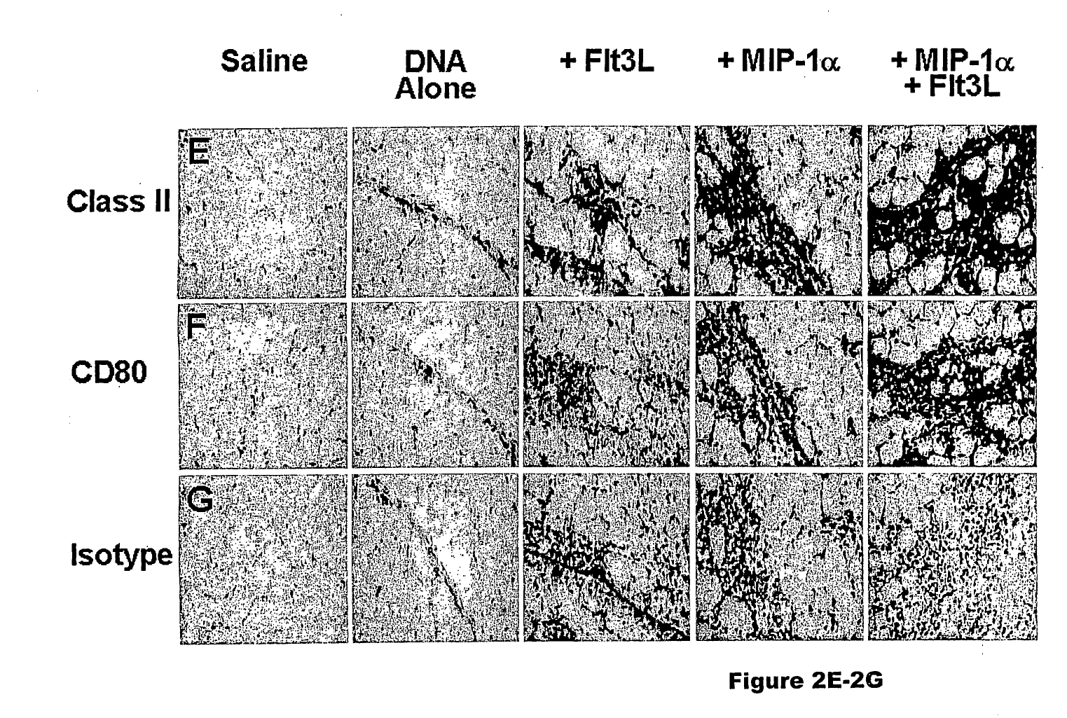

The nature of these cellular infiltrates was assessed by single-color immunohistochemistry. Acetone-fixed 5 μm sections were first treated with 0.5% hydrogen peroxide in phosphate-buffered saline (PBS) for 15 min to quench endogenous peroxidase. The sections were then washed with PBS, and free biotin was blocked. Sections were then incubated with the primary antibodies at room temperature for one hour. Monoclonal antibodies were labeled with biotin. After incubation, the slides were washed three times with PBS and developed. As depicted in FIG. 2A, none of these sections stained positively for CD3, indicating that the infiltrates contained few CD3⁺ T lymphocytes. In contrast, as shown in FIG. 2B, we observed substantial differences in CD11b staining among these sections, reflecting variable numbers of CD11b⁺ macrophages or dendritic cells recruited by the various vaccine regimens. Muscle sections from mice immunized with the DNA vaccine alone contained few CD11b⁺cells. Plasmid Flt3L recruited limited numbers of additional CD11b⁺ cells, indicating small but distinct populations of antigen-presenting cells within heterogeneous cellular infiltrates. Plasmid MIP-1α and the combination of both plasmid MIP-1α and plasmid Flt3L recruited large infiltrates consisting predominantly of CD11b⁺ cells, suggesting that plasmid MIP-1α exerted a specific chemotactic effect that recruited antigen-presenting cells at the site of inoculation. Although CD11b is also expressed on NK cells and granulocytes, it is likely that these cells were not present in large numbers based on subsequently demonstrated staining patterns.

FIGS. 2C-D show the extent of DC recruitment in these sections using mAbs specific for the DC-specific markers S100 and CD83. The DNA vaccine alone recruited few S100⁺ DCs to the injection site. In contrast, moderate numbers of S100⁺ DCs were recruited by plasmid Flt3L alone and plasmid MIP-1α alone. Staining for the DC maturation marker CD83 was low to moderate in these sections, indicating that these DCs had predominantly an immature phenotype. Interestingly, the combination of both plasmid cytokines resulted in massive infiltrates of S100⁺ DCs that also exhibited high levels of CD83 expression. All sections showed minimal staining for the macrophage-specific marker F4/80.

The activation state of the DCs recruited by these vaccine modalities was determined by assessing MHC class II and CD80 expression. As shown in FIGS. 2E-F, the cells recruited by plasmid Flt3L alone or plasmid MIP-1α alone exhibited low to moderate levels of MHC class II and CD80 expression. In contrast, cellular infiltrates recruited by the combination of both plasmid cytokines exhibited high levels of MHC class II and CD80 expression, consistent with a highly activated phenotype. Staining of these sections with an isotype control mAb was negative (FIG. 2G).

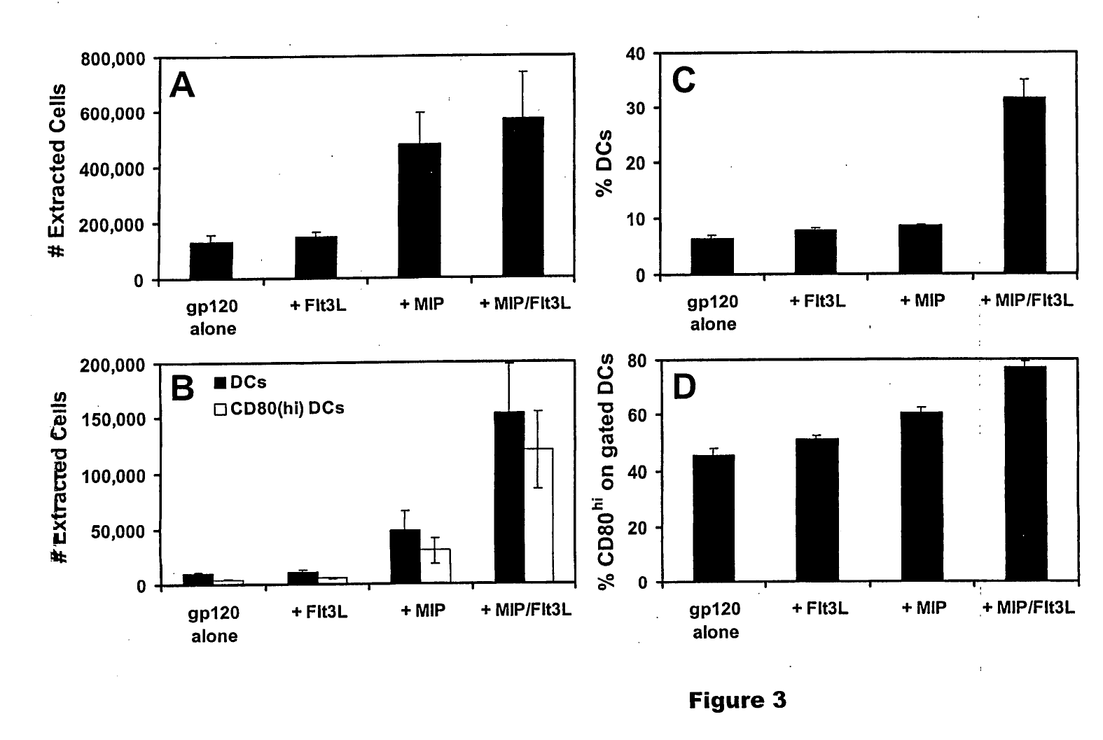

To analyze these cellular infiltrates in greater detail, muscles were excised from similarly immunized mice on day 7 after injection (n=8/group), homogenized, and digested with collagenase and trypsin. Cell suspensions were then assessed for DCs by staining with mAbs and four-color flow cytometric analysis. As shown in FIG. 3A, 5-fold more total cells were extracted from muscles injected with plasmid MIP-1α or both plasmid cytokines as compared with muscles injected with the DNA vaccine alone. As depicted in FIG. 3B, muscles injected with plasmid MIP-1α also had 5-fold more gated CD3⁻CD19⁻ class II⁺CD11c⁺DCs and 6-fold more activated CD80^{hi}DCs as compared with muscles injected with the DNA vaccine alone. Interestingly, muscles injected with both plasmid cytokines had 16-fold more DCs and 27-fold more activated CD80^{hi} DCs as compared with muscles injected with the DNA vaccine alone, consistent with the results observed by immunohistochemistry. These data demonstrate that plasmid MIP-1α and plasmid Flt3L exert synergistic effects that substantially exceed their additive individual effects.

The large numbers of DCs found in muscles injected with both plasmid cytokines reflected not only a larger number of infiltrating cells but also a higher percentage of DCs (32%) in these infiltrates as compared with the infiltrates observed in the other groups (6-8%) (FIG. 3C). Moreover, 77% of DCs extracted from muscles injected with both plasmid cytokines exhibited high levels of CD80 expression as compared with 60% from muscles injected with plasmid MIP-1α, 51% from muscles injected with plasmid Flt3L, and 41% from muscles injected with the DNA vaccine alone (FIG. 3D). These results demonstrate that plasmid MIP-1α alone is more effective than plasmid Flt3L alone in recruiting DCs to the injection site. When these plasmid cytokines are administered together, it is likely that plasmid Flt3L expands and matures the DC populations recruited by plasmid MIP-1α, thereby resulting in large numbers of mature DCs at the site of inoculation. Similar recruitment and activation of DCs were observed when the plasmid cytokines were inoculated without the DNA vaccine.

### EXAMPLE 2

### MIP-1α, Flt3L, and GM-CSF Synergistically Increase DNA Vaccine Immune Response

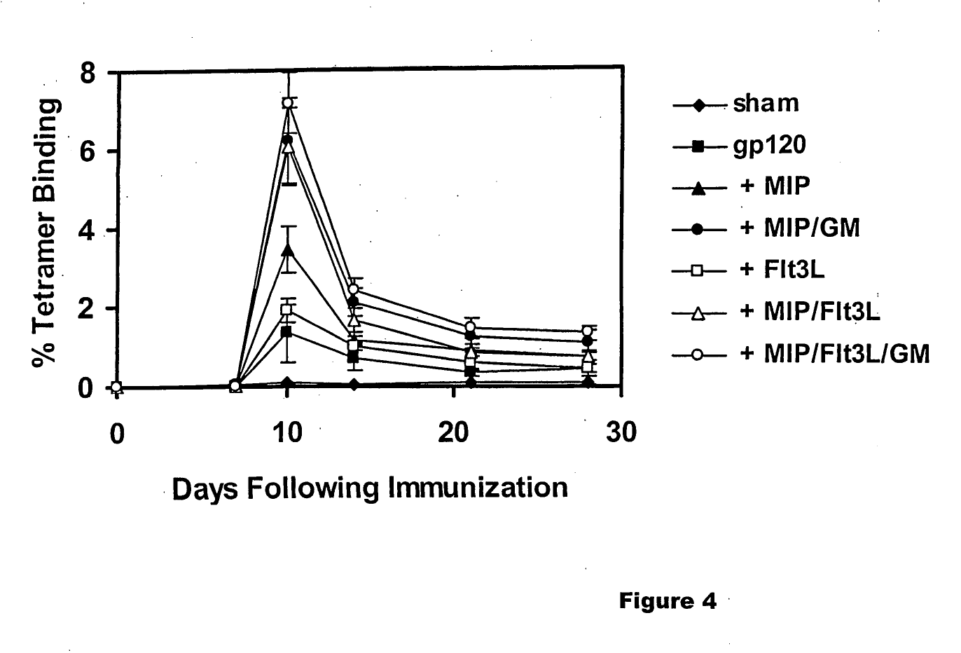

The ability of Flt3L, GM-CSF, and MIP-1α to augment T cell responses elicited by a DNA vaccine was investigated in mice, using a model vaccine encoding the HIV-1 Env IIIB gp120 protein. Balb/c mice were immunized with: a sham plasmid vaccine or a gp120 plasmid vaccine, which was administered alone or in combination with MIP-1α; MIP-1α and GM-CSF; Flt3L; MIP-1α and Flt3L; MIP-1α, Flt3L, and GM-CSF (FIG. 4). Each of these adjuvants was delivered by means of a plasmid.

Mice were primed intramuscularly with sham plasmid DNA, gp120 DNA vaccine alone, or gp120 DNA vaccine with or without plasmid MIP-1α and Flt3L. 50 μg of each plasmid was administered with sufficient sham plasmid DNA to keep the total DNA dose constant (e.g., at 150 μg DNA per animal). All plasmids were mixed together and delivered as 50 μl injections in the quadriceps. At week 8, mice were boosted with 50 μg sham plasmid DNA or 50 μg gp120 DNA vaccine alone. The immune responses of mice were assessed after the primary immunization (weeks 1-8) and after the boost immunization (weeks 9-16). Mice were therefore bled at weeks 0, 1, 2, 3, 4, 6, 8, 9, 10, 11, 12, 14, and 16 following primary injection. Vaccine-elicited CD8⁺ T lymphocyte responses were monitored by D^{d}/P 18 tetramer binding, and vaccine-elicited antibody responses were monitored by anti-gp120 ELISAs as described above. At week 16, mice were sacrificed, and splenocytes were utilized in IFN-γ ELISPOT assays, proliferation assays, and chromium release cytotoxicity assays.

FIG. 4 shows that the inoculation with the HIV-1 gp120 DNA vaccine in combination with MIP-1α and Flt3L (in the presence or absence of GM-CSF) resulted in the augmentation of CD8+ T cell responses as measured by D^{d}/P18 tetramer assays in mice. The combination of MIP-1α and Flt3L resulted in a synergistic adjuvant effect as this combination resulted in a greater adjuvant effect than the sum of the adjuvant effects of MIP-1α or Flt3L delivered alone. This potent immune response may result from MIP-1α recruiting large numbers of dendritic cells to the site of inoculation, where Flt3L induces their maturation, activation, and proliferation. The immune response resulting from this particular combination was comparable to that of the combination of MIP-1α and GM-CSF. The combination of all three adjuvants, namely MIP-1α, Flt3L, and GM-CSF, resulted in the greatest immunogenicity. This synergistic effect may be attributed the contribution of each of these three factors in distinct steps in the recruitment and activation of APCs, namely the dendritic cell chemotactic properties of MIP-1α, the proliferative and activation effects of Flt3L, and the macrophage chemotactic properties, maturation signals, and augmented CD4⁺ T cell help afforded by GM-CSF.

### EXAMPLE 3

### Recruitment of DCs Augments DNA Vaccine Immunogenicity

Groups of mice (n=8/group) were immunized with sham plasmid, the gp120 DNA vaccine alone, or the gp120 DNA vaccine with plasmid MIP-1α, plasmid Flt3L, or the combination of both plasmid cytokines. 50 μg of each plasmid was inoculated with sufficient sham plasmid to keep the total dose of DNA per animal constant.

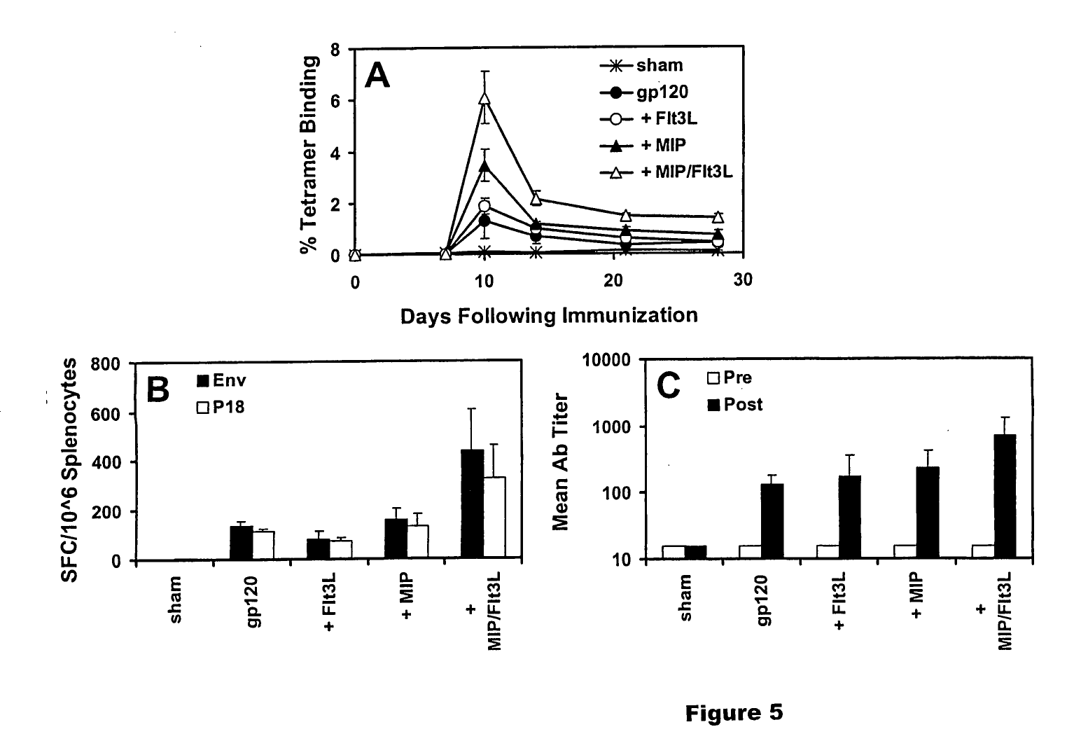

Vaccine-elicited CD8⁺ T lymphocyte responses specific for the immunodominant H-2D^{d}-restricted P18 epitope (RGPGRAFVTI) (Takahashi et al., Science 255:333-336 (1992)) were assessed at various time points following immunization by tetramer binding to CD8⁺ T lymphocytes isolated from peripheral blood (Barouch et al., J. Immunol. 168:562-568 (2002); Barouch et al., J. Virol. 77:8729-8735 (2003); Altman et al., Science 274:94-96 (1996)). As demonstrated in FIG. 5A, following a single injection of the unadjuvanted gp120 DNA vaccine, mice developed peak tetramer⁺CD8⁺ T lymphocyte responses of 1.3% on day 10 following immunization. These responses declined to 0.4% by day 28. Addition of plasmid Flt3L had minimal effects on the kinetics or magnitudes of these responses. In contrast, mice that received the DNA vaccine with plasmid MIP-1α developed higher peak tetramer⁺CD8⁺ T lymphocyte responses of 3.4% on day 10 following immunization. This augmentation was transient and memory tetramer⁺CD8⁺ T lymphocyte responses in these mice were indistinguishable from those elicited by the unadjuvanted DNA vaccine by day 28. Administering higher doses of plasmid MIP-1α did not further augment these responses. Importantly, mice that received the DNA vaccine with both plasmid MIP-1α and plasmid Flt3L developed 5-fold higher peak tetramer⁺CD8⁺ T lymphocyte responses of 6.1% on day 10 and maintained 3-fold higher memory responses of 1.3% by day 28. These responses were significantly higher than those elicited by the unadjuvanted DNA vaccine (P<0.001 comparing groups on day 10 or day 28 using analyses of variance with Bonferroni adjustments to account for multiple comparisons). Tetramer⁺CD8⁺ T lymphocyte responses in lymph nodes were comparable with the responses observed in peripheral blood. Thus, coadministration of the combination of plasmid MIP-1α and plasmid Flt3L results in a synergistic and durable enhancement of DNA vaccine-elicited CD8⁺ T lymphocyte responses.

Vaccine-elicited cellular immune responses were also assessed by IFN-γ ELISPOT assays using splenocytes harvested on day 28 following immunization and stimulated with a pool of overlapping Env peptides or the P18 epitope peptide. As shown in FIG. 5B, vaccine-elicited ELISPOT responses were not detectably augmented by plasmid Flt3L alone or plasmid MIP-1α alone. Consistent with the tetramer binding assays, mice that received the DNA vaccine with both plasmid Flt3L and plasmid MIP-1α exhibited substantially increased Env-specific and P18-specific ELISPOT responses as compared with mice that received the DNA vaccine alone (P<0.001). As demonstrated in FIG. 5C, Env-specific antibody responses as measured by ELISA were also significantly augmented by these plasmid cytokines (P<0.01). These data show that the recruitment, expansion, and activation of DCs at the site of inoculation using plasmid MIP-1α and plasmid Flt3L markedly enhances the magnitude and durability of DNA vaccine-elicited cellular and humoral immune responses.

### EXAMPLE 4

### MIP-3α , Flt3L, and GM-CSF Synergistically Increase DNA Vaccine Immune Response

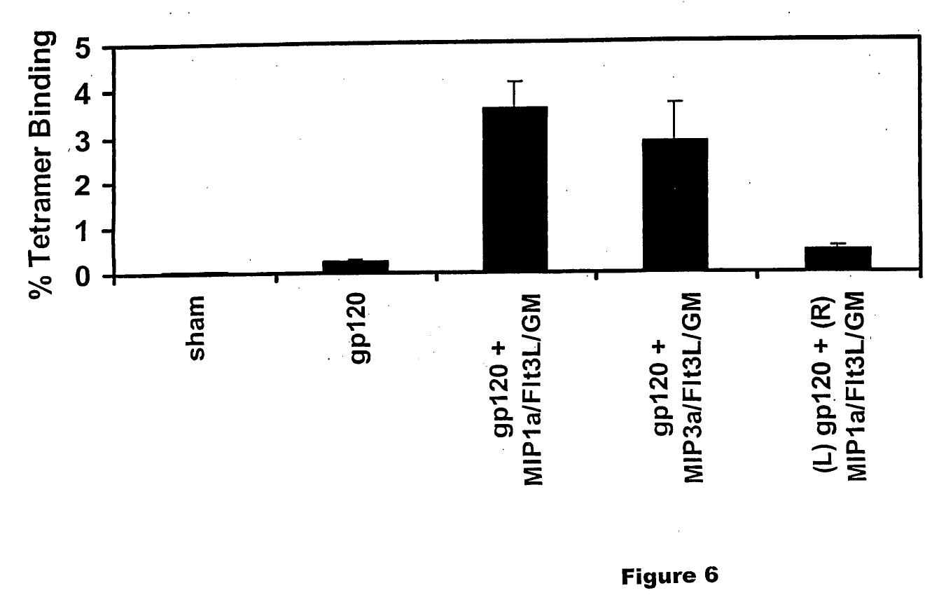

We next investigated whether the plasmid chemokine MIP-3α could also augment the immune response elicited by a DNA vaccine when administered with Flt3L. Balb/c mice were immunized with: a sham plasmid vaccine or a HIV-1 Env IIIB gp120 plasmid vaccine, which was administered alone or in combination with MIP-1α, Flt3L, and GM-CSF, or MIP-3α, Flt3L, and GM-CSF. Each of these adjuvants was delivered by means of a plasmid. As in Example 2, 50 μg of each plasmid was administered with sufficient sham plasmid DNA to keep the total DNA dose constant. All plasmids were mixed together and delivered as 50 μl injections in the quadriceps. Vaccine-elicited CD8⁺ T lymphocyte responses were monitored by D^{d}/P18 tetramer binding as described above. As shown in FIG. 6, plasmid MIP-3α is nearly identical to MIP-1α in its effectiveness in augmenting the T cell response elicited by a DNA vaccine. The administration of MIP-3α, Flt3L, and GM-CSF together with the gp120 vaccine resulted in a synergistic T cell response.

### EXAMPLE 5

### The Synergistic Effects of Plasmid MIP-1α and Plasmid Flt3L on DNA Vaccine Immunogenicity Is Generalizable

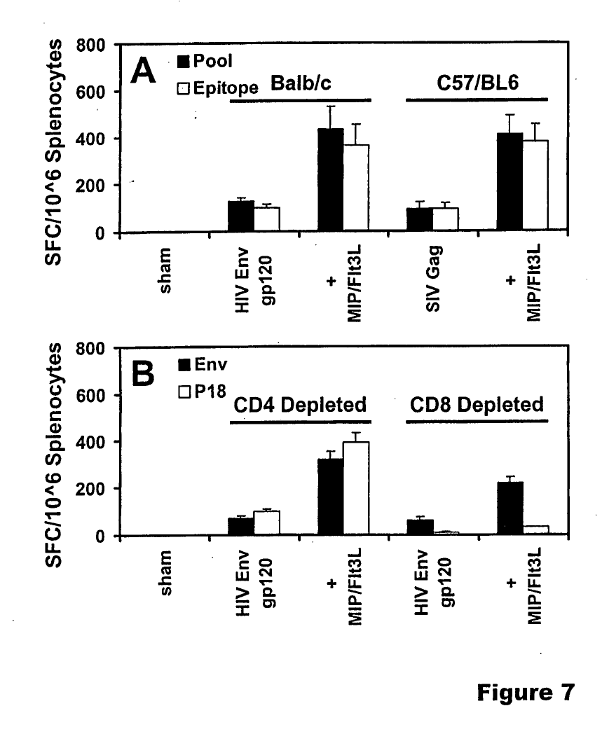

To explore the generalizability of the adjuvant effects of plasmid MIP-1α and plasmid Flt3L, we assessed cellular immune responses elicited by the HIV-1 Env gp120 DNA vaccine in Balb/c mice and by the SIVmac239 Gag DNA vaccine in C57/BL6 mice. As shown in FIG. 7A, coadministration of plasmid MIP-1α and plasmid Flt3L augmented both pooled peptide and dominant epitope-specific ELISPOT responses in both systems using unfractionated splenocytes, demonstrating that the observed adjuvant effects are neither antigen-specific nor strain-specific. Moreover, as depicted in FIG. 5B, plasmid MIP-1α and plasmid Flt3L augmented both CD8⁺ and CD4⁺ T lymphocyte responses as measured by ELISPOT assays using fractionated splenocyte populations from Balb/c mice.

### EXAMPLE 6

### Expansion of Primary Immune Responses Following Boost Immunization

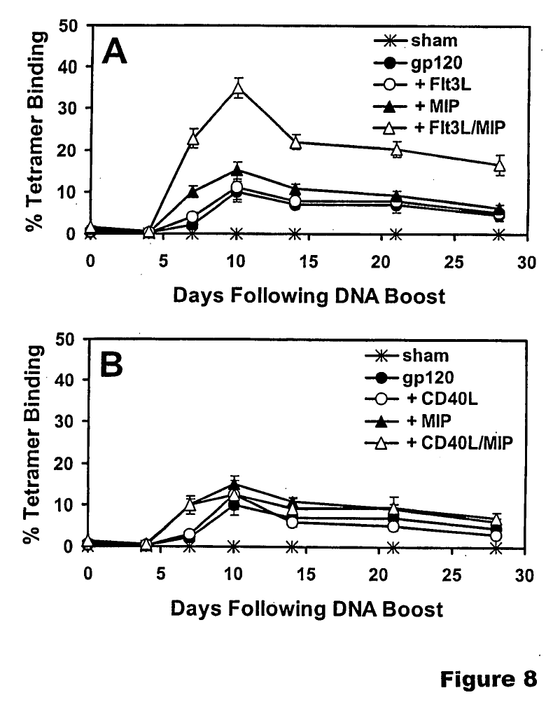

The ability of primary immune responses to expand following re-exposure to antigen was assessed. In the first experiment, groups of mice were primed as described in Example 1 with the gp120 DNA vaccine alone or with plasmid Flt3L, plasmid MIP-1α, or both plasmid Flt3L and plasmid MIP-1α. At week 6 following primary immunization, all groups of vaccinated mice were boosted with 50 μg gp120 DNA vaccine alone to expand the memory T lymphocyte responses primed by the various vaccine regimens. As shown in FIG. 8A, mice primed with the unadjuvanted DNA vaccine developed peak secondary tetramer⁺CD8⁺ T lymphocyte responses of 10.2% on day 10 following the boost immunization. These responses were not detectably augmented by plasmid Flt3L and were only marginally enhanced by including plasmid MIP-1α in the priming regimen. Strikingly, mice that were primed with the DNA vaccine with both plasmid MIP-1α and plasmid Flt3L exhibited peak tetramer⁺CD8⁺ T lymphocyte responses of 34.9% following the boost immunization, demonstrating the substantial potential of memory CD8⁺ T lymphocytes in these mice to expand rapidly following a boost immunization. As depicted in FIG. 8B, substituting a plasmid expressing the costimulatory molecule CD40L in place of plasmid Flt3L abrogated these adjuvant effects. Thus, plasmid MIP-1α requires plasmid Flt3L for synergy, presumably reflecting the ability of Flt3L to expand and mature DCs.

In another experiment, mice were primed with the gp120 DNA vaccine, with or without adjuvants and were subsequently boosted with rAd5-Env. Mice were primed with sham plasmid DNA, gp120 DNA vaccine alone, or gp120 DNA vaccine with various combinations of plasmid MIP-1α, plasmid Flt3L, and plasmid GM-CSF. 50 μg of each plasmid was administered with sufficient sham plasmid DNA to keep the total inoculum of DNA constant at 200 μg per animal. All plasmids were mixed together and delivered as 50 μl injections in the quadriceps. At week 8, mice were boosted with 10⁶ particles sham nonrecombinant Ad5 or 10⁶ particles rAd5-Env IIIB gp140, as described above. Mice were bled at weeks 0, 1, 2, 3, 4, 6, 8, 9, 10, 11, 12, 14 and 16, and vaccine-elicited immune responses were monitored by D^{d}/P18 tetramer binding and anti-gp120 ELISAs. At week 16, splenocytes were utilized for functional IFN-γ ELISPOT, proliferation, and chromium release cytotoxicity-assays. Multiple comparisons between various test groups were achieved employing Wilcoxon rank-sum tests with Bonferroni adjustments to account for multiple comparisons.

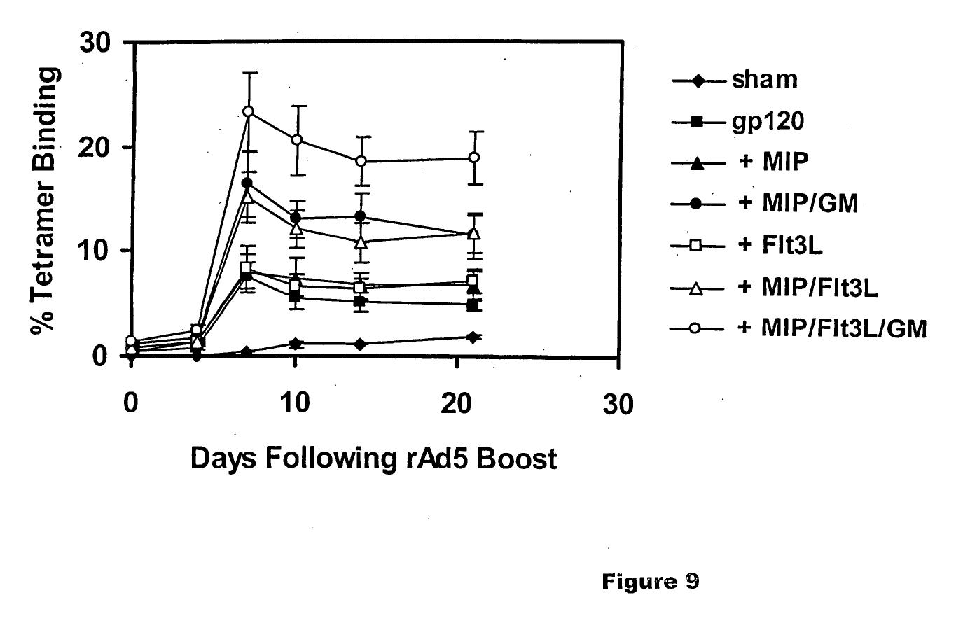

As shown in FIG. 9, MIP-1α and Flt3L (delivered by means of plasmids) during DNA priming resulted in markedly increased responses following the rAd5 boost. The immunogenicity of this combination was comparable to that achieved with the combination of MIP-1α and GM-CSF. The highest responses were observed in mice that were administered the combination of MIP-1α, Flt3L, and GM-CSF.

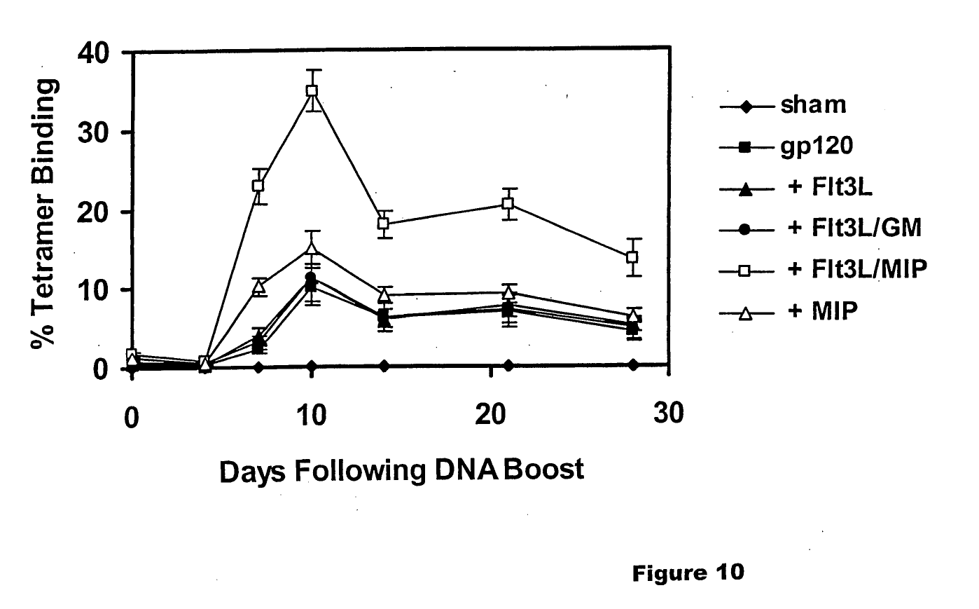

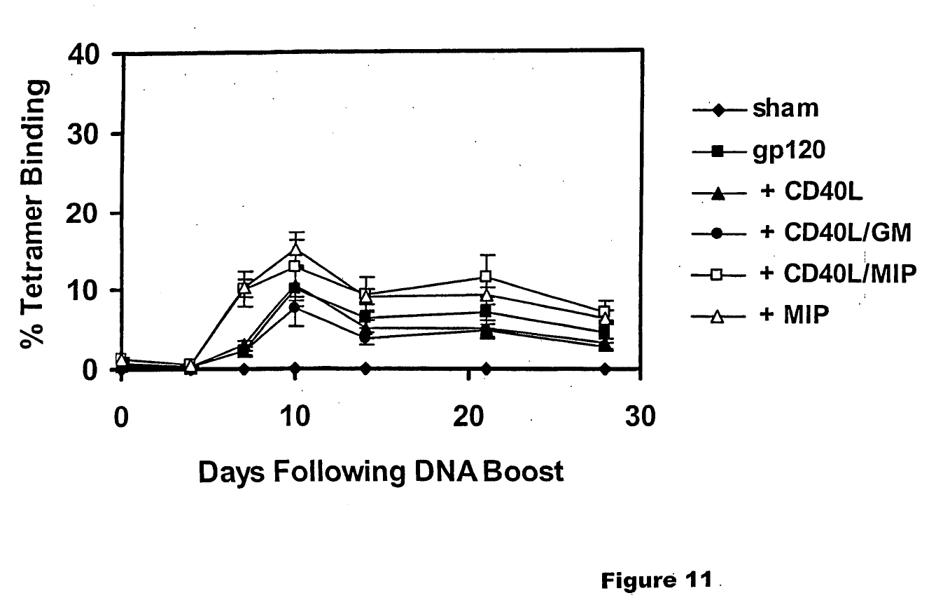

Mice were next primed with the gp120 DNA vaccine with or without adjuvants and subsequently boosted with the gp120 DNA vaccine alone (see FIG. 10). The combination of MIP-1α and Flt3L administered during plasmid DNA priming markedly increased the immune response following the plasmid DNA boost. We next sought to determine whether this adjuvancy was a general effect and therefore tested the ability of the CD40 ligand (CD40L) to enhance the immunogenicity of a DNA vaccine co-injected with MIP-1α. CD40L (CD154) is a co-stimulatory molecule expressed on activated T lymphocytes that interacts with CD40 and activates APCs. As shown in FIG. 11, adjuvant regimens involving the administration of CD40L in place of Flt3L were much less effective and did not substantially augment the immunogenicity of the vaccines.

### EXAMPLE 7

### Mechanistic Studies of Plasmid MIP-1α and Plasmid Flt3L Adjuvanticity

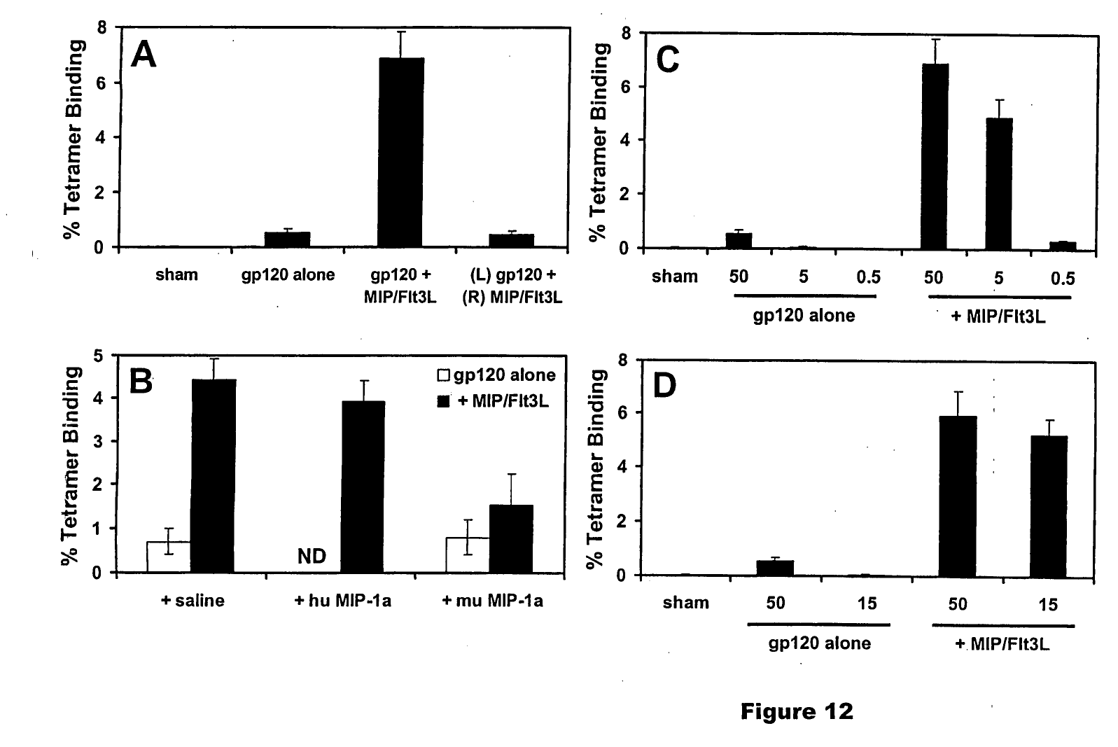

Without wishing to be bound by any particular mechanism, we believe that plasmid MIP-1α and Flt3L function by exerting local effects and in particular by recruiting, expanding, and activating DCs at the site of inoculation and antigen production. To investigate this, we first assessed the effects of separating the DNA vaccine and the plasmid cytokines into different muscle groups. Mice were immunized with either 50 μg gp120 DNA vaccine and 50 μg of each plasmid cytokine mixed together and delivered equally in both legs, or 50 μg gp120 DNA vaccine in the left leg and 50 μg of each plasmid cytokine in the right leg. Interestingly, as shown in FIG. 12A, anatomic separation of the DNA vaccine and the plasmid cytokines completely abrogated the adjuvanticity of plasmid MIP-1α and plasmid Flt3L. Thus, these plasmid cytokines exert predominantly local effects at the site of antigen production.

We next assessed the effects of disrupting the chemokine gradient established by intramuscular injection of plasmid MIP-1α by administering high-dose, systemic MIP-1α protein. Since chemotaxis is dependent on an intact chemokine gradient rather than absolute chemokine concentrations, we sought to investigate whether disrupting the chemokine gradient would effectively block DC recruitment and abrogate the adjuvanticity of these plasmid cytokines. Mice were immunized with the gp120 DNA vaccine alone or mixed with plasmid MIP-1α and plasmid Flt3L and also received daily injections of either saline or 1 μg recombinant MIP-1α protein administered i.v. and i.p. We estimate that this dose of recombinant MIP-1α protein exceeded the amount expressed by the plasmid by >1000-fold (Barouch et al., Vaccine 22:3092-3097 (2004)). High-dose systemic administration of murine MIP-1α protein reduced DC recruitment by >90%. Accordingly, as shown in FIG. 12B, inhibiting DC recruitment also markedly suppressed the adjuvanticity of these plasmid cytokines. These data confirm that the adjuvanticity of plasmid MIP-1α and plasmid Flt3L requires active DC recruitment to the site of inoculation by an intact chemokine gradient.

### EXAMPLE 8

### Specific Chemotaxis of Dendritic Cells is Substantially More Effective than Nonspecific Inflammation in Priming Immune Responses

Intramuscular administration of unadjuvanted DNA vaccines typically requires high doses (50 μg) and large injection volumes (50 μl) to elicit immune responses in mice. The nonspecific inflammation that occurs at the site of inoculation as a result of these injection parameters likely provides a limited number of antigen-presenting cells that are able to prime low frequency immune responses. We found that lowering the vaccine dose or the injection volume substantially reduced this inflammation and abrogated vaccine-elicited immune responses, depicted in FIGS. 12C-D. Interestingly, lowering the dose of the MIP-1α/Flt3L-augmented DNA vaccine from 50 μg to 5 μg of each plasmid or lowering the injection volume from 50 μl to 15 μl had minimal effects on vaccine-elicited tetramer⁺CD8⁺ T lymphocyte responses. These data suggest that specific chemotaxis of DCs is substantially more effective than nonspecific inflammation in recruiting DCs to the site of inoculation and in priming immune responses under these limiting conditions.

### EXAMPLE 9

### Recruitment of DCs Enhances the Protective Efficacy of DNA Vaccines

To confirm the functional significance of the DNA vaccine-elicited immune responses, we assessed the protective efficacy of these various vaccine regimens against challenge with recombinant vaccinia virus expressing HIV-1 IIIB Env. Groups of mice (n=4/group) were immunized with sham plasmid, the gp120 DNA vaccine, or the gp120 DNA vaccine with plasmid MIP-1α and plasmid Flt3L. 50 μg of each plasmid was administered with sufficient sham plasmid to keep the total DNA dose per animal constant. At week 12, mice were challenged i.p. with 10⁷ pfu recombinant replication-competent vaccinia expressing HIV-1 Env IIIB.

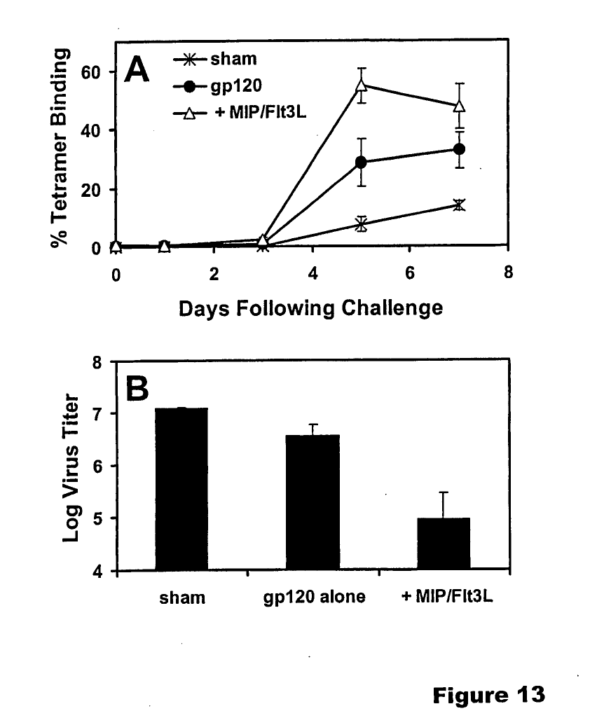

Following challenge, we observed anamnestic tetramer⁺CD8⁺ T lymphocyte responses in the DNA vaccinated mice as compared with the mice that received the sham plasmid (FIG. 13A). Secondary responses were substantially higher in the mice primed with the MIP-1α/Flt3L-augmented DNA vaccine as compared with mice primed with the unadjuvanted DNA vaccine. Importantly, as shown in FIG. 13B, the MIP-1α/Flt3L-augmented DNA vaccine afforded a 2.1 log reduction of vaccinia virus titers in ovaries harvested on day 7 following challenge as compared with sham vaccinated mice (P<0.001 comparing groups using analyses of variance with Bonferroni adjustments). In contrast, the unadjuvanted DNA vaccine afforded only a 0.5 log reduction in vaccinia virus titers as compared with sham vaccinated mice, reflecting the high stringency of this viral challenge (P>0.05). Thus, the MIP-1α/Flt3L-augmented DNA vaccine elicited higher pre-challenge primary CD8⁺ T lymphocyte responses, higher post-challenge anamnestic CD8⁺ T lymphocyte responses, and improved protective efficacy against a recombinant vaccinia virus challenge as compared with the unadjuvanted DNA vaccine. These studies confirm the functional significance of the enhanced immunogenicity afforded by plasmid MIP-1α and plasmid Flt3L.

### EXAMPLE 10

### Immunogenicity and Protective Efficacy of Cytokine-Augmented DNA Vaccine Priming Followed by rAd5 Boosting in Rhesus Monkeys with Pre-Existing Anti-Ad5 Immunity

Candidate AIDS vaccines utilizing DNA prime/rAd5 boost approaches have demonstrated impressive immunogenicity in rhesus monkeys and are currently entering large-scale clinical trials. The clinical utility of rAd5-based HIV-1 vaccines, however, is likely to be limited by the high prevalence of pre-existing anti-Ads immunity present in human populations. In fact, early data from phase 1 clinical studies suggests that immune responses elicited by rAd5 vectors in humans are in fact substantially blunted by pre-existing anti-Ad5 immunity. Our studies similarly showed that anti-Ad5 immunity dramatically inhibited the immunogenicity of rAd5 in mice. In mice with anti-Ad5 immunity, unadjuvanted DNA vaccine priming followed by rAd5 boosting elicited only marginal immune responses. In contrast, GM-CSF/IP-1α-augmented DNA vaccine priming followed by rAd5 boosting generated potent immune responses in mice with anti-Ad5 immunity. These data demonstrate that augmenting DNA vaccine primigusing plasmid cytokine adjuvants may represent a useful strategy to increase the overall immunogenicity of DNA prime/rAd5 boost vaccine strategies and to compensate in part for the inhibitory effects of pre-existing anti-vector immunity.

To investigate the hypothesis that increasing the efficiency of DNA vaccine priming using plasmid cytokine adjuvants increases the immunogenicity of DNA prime/rAd5 boost vaccine regimens and partially overcome the inhibitory effects of anti-Ad5 immunity in rhesus monkeys, the immunogenicity of DNA vaccine priming with the MIP-1α and Flt3L was determined. The ability of these plasmid cytokine adjuvants to improve the efficiency of rAd5 boosts in monkeys with anti-Ad5 immunity and to enhance protective efficacy against a pathogenic, heterologous SIV challenge may be assessed as follows.

18 adult Mamu-A*01-negative rhesus monkeys are utilized since this MHC class I allele has been shown to affect disease courses following infection with SIVmac251, SIVmac239, and SHIV-89.6P. Monkeys are inoculated with nonrecombinant sham Ad5 to induce anti-Ad5 immunity, primed with DNA vaccines with or without plasmid cytokine adjuvants (MIP-1α and Flt3L), boosted with rAd5 vectors, and then challenged with SIVsmE660 as follows:

| Group | N | Weeks −16, −8 | Weeks 0, 4, 8 | Week 24 | Week 36 |
|---|---|---|---|---|---|
| 1 | 6 | Sham Ad5 | Sham Plasmid DNA | Sham Ad5 | SIVsmE660 i.v. |
| 2 | 6 | Sham Ad5 | DNA Vaccines Alone | rAd5 | SIVsmE660 i.v. |
| 3 | 6 | Sham Ad5 | DNA Vaccines + Plasmid | rAd5 | SIVsmE660 i.v. |
| | | | MIP-1α + Plasmid Flt3L | | |

Plasmid DNA vaccines and rAd5 vaccines expressing the SIVmac239 env or gag-pol-nef genes may be utilized. The sham plasmid DNA that may be used includes the pVRC plasmid without any insert, and the sham Ad5 may be nonrecombinant Ad5. Rhesus Flt3L cDNA may be amplified by polymerase chain reaction from mRNA isolated from rhesus PBMC using primers specific for human Flt3L. This procedure was successful in cloning bovine Flt3L. The cloning of rhesus Flt3L is standard in the art particularly since human, bovine, and murine Flt3L cDNA exhibit a high degree (72-81%) of sequence homology. Rhesus Flt3L clones may be sequenced and subcloned into the pVRC expression plasmid. Expression is confirmed by transient transfections of 293 cells followed by ELISA analyses of culture supernatants using a human Flt3L ELISA. Bioactivity of culture supernatants containing rhesus Flt3L may be confirmed by their ability to expand dendritic cells, which has been described as a bioassay for human and murine Flt3L.

All monkeys are pre-immunized with 10¹¹ particles nonrecombinant Ad5 16 and 8 weeks prior to primary immunization to induce active anti-Ad5 immunity. At weeks 0, 4, and 8, the control animals in Group 1 receive 10 mg sham plasmid DNA. Animals in Group 2 will receive 2.5 mg env DNA vaccine, 2.5 mg gag-pol-nef DNA vaccine, and 5 mg sham plasmid DNA at these time points. Animals in Group 3 receive 2.5 mg env DNA vaccine, 2.5 mg gag-pol-nef DNA vaccine, and 2.5 mg of each plasmid cytokine at these time points. All plasmids are mixed and co-delivered intramuscularly as two 1 ml injections, one in each quadriceps, by Biojector inoculation. At week 24, monkeys are boosted intramuscularly with 2×10¹¹ particles nonrecombinant empty Ad5 (Group 1) or 10¹¹ particles rAd5-env and 10¹¹ particles rAd5-gag-pol-nef (Groups 2 and 3).

To induce active anti-Ad5 immunity, monkeys receive 10¹¹ particles nonrecombinant Ad5 16 and 8 weeks prior to primary immunization. To measure the magnitude of anti-vector immune responses, serum is collected every two weeks and tested for anti-Ad5 neutralizing antibody responses. Ad5 neutralization assays are performed by assessing the ability of serum dilutions to block infection of A549 cells by Ad5-luciferase reporter constructs. Optimally, anti-Ad5 90% neutralizing antibody titers should reach 200-1000, which are titers typically found in humans. If these titers are observed six weeks after a single injection of Ad5, then the second Ad5 injection is cancelled. If these titers are not achieved six weeks after the second injection of Ad5, then additional Ads injections are performed and the DNA priming is delayed.

The magnitude and breadth of vaccine-elicited cellular immune responses are monitored at weeks 0, 2, 4, 6, 8, 10, 12, 16, 20, 24, 26, 28, 30, 32, 34, and 36 following primary immunization 20 mls EDTA-anticoagulated blood is obtained at each time point from each animal, and PBMCs are utilized in pooled peptide IFN-γ ELISPOT assays specific for Gag, Pol, Nef, and Env. At weeks 10, 24, 26, and 36, CD4-depleted PBMCs and CD8-depleted PBMCs are used in similar ELISPOT assays to assess fractionated CD8⁺ and CD4⁺ T cell responses. IFN-γ intracellular cytokine staining (ICS) assays using similar peptide pools as well as Gag- and Env-specific proliferation assays are also performed at these time points. Humoral immune responses against Gag and Env are monitored by ELISA, and virus neutralization assays against SIVmac239 and SIVsmE660 may also be performed. Anti-Ad5 neutralizing antibody titers in these animals may also be monitored at each time point.

The magnitude, breadth, kinetics, and durability of immune responses elicited in Group 2 may also be compared with those elicited in Group 3. Peak and memory ELISPOT, ICS, and neutralizing antibody responses at weeks 10, 24,26, and 36 may be compared between Groups 2 and 3 using Wilcoxon rank-sum tests. The augmentation of DNA vaccine-primed immune responses at weeks 10 and 24 by MIP-1α and Flt3L confirms the strategy of utilizing one plasmid to recruit dendritic cells and a second plasmid to induce proliferation and activation of these dendritic cells. Suboptimal results may be due to inadequate doses of plasmids, low in vivo expression levels, or reduced intrinsic responsiveness of monkeys as compared with mice to these cytokines. The optimization of such strategies may involve using higher doses of plasmid MIP-1α and plasmid Flt3L.

Marginal increases of vaccine-elicited immune responses following the rAd5 boost in monkeys in Group 2 are typically-observed as a result of the inhibitory effects of pre-existing anti-Ad5 immunity. Our studies demonstrated a dramatic >90% inhibitory effect of anti-Ad5 immunity on the subsequent immunogenicity of rAd5 boosts in mice. If no responses are observed following the rAd5 boost in this study, a lower dose of Ad5 may be utilized for pre-immunization. If potent responses are observed following the rAd5 boost, higher doses of Ad5 may be used for pre-immunization. Our studies also showed that mice with anti-Ad5 immunity that were primed with DNA vaccines alone generated only marginal responses following the rAd5 boost, whereas mice with anti-Ad5 immunity that were primed with cytokine-augmented DNA vaccines generated potent responses following the same rAd5 boost. Accordingly, the efficiency of DNA vaccine priming may be critical in determining the magnitude of immune responses following rAd5 boosts, particularly in the limiting setting of anti-vector immunity. Monkeys in Group 3 may therefore exhibit markedly higher immune responses than animals in Group 2 following the rAd5 boost at weeks 26 and 36. Such a result demonstrates the potential utility of plasmid MIP-1α nd plasmid Flt3L to enhance the immunogenicity of DNA prime/rAd5 boost regimens in rhesus monkeys with pre-existing anti-Ad5 immunity. However, the lack of differences in immune responses between Groups 2 and 3 prior to the rAd5 boost does not predict that differences would emerge following the boost.

To assess the protective efficacy of vaccine-elicited immune responses, all monkeys at week 36 are challenged intravenously with 100 MID₅₀ SIVsmE660. This is an extremely stringent challenge, since it is a heterologous SIV challenge and since the vaccine-elicited immune responses is likely be blunted as a result of pre-existing anti-Ad5 immunity. The breadth and magnitude of the anamnestic cellular immune responses is first examined by ELISPOT assays using Gag, Pol, Nef, and Env peptide pools at weeks 0, 1, 2, 3, 4, 6, 8, 10, 12, 16, 20, 24, 28, 32, 36, 40,44, 48, and 52 following challenge. CD4-depleted ELISPOT assays, CD8-depleted ELISPOT assays, ICS assays, and proliferation assays are also performed at selected time points. The emergence of neutralizing antibody responses against SIVsmE660 is also measured. The vaccinated animals in Groups 2 and 3 typically exhibit more rapid and more potent anamnestic immune responses following challenge as compared with the control animals in Group 1. However, if higher immune responses are observed in the animals in Group 3 as compared with Group 2 prior to challenge, then these differences may be maintained following challenge. However, if similar immune responses are observed in Groups 2 and 3 prior to challenge, then similar secondary immune responses should be observed following challenge.

CD4⁺ T lymphocyte counts and plasma viral RNA levels (Bayer Diagnostics) were next monitored in these animals. Comparisons of CD4⁺ T lymphocyte counts and plasma viral RNA levels at viral setpoint are performed among all three groups using Wilcoxon rank-sum tests with Bonferroni adjustments to account for multiple comparisons. The immune correlates of protection are also studied by assessing whether peak or memory cellular immune responses prior to challenge correlate with setpoint plasma viral RNA levels following challenge using Spearman rank correlation tests. The overall control of viral replication may not be considerably impressive as a result of lower magnitude immune responses following the rAd5 boost. Nevertheless, a partial attenuation of viral replication in the vaccinated animals is typical, since an SIVmac239 gag DNA vaccine alone has been shown to provide partial control of a heterologous SIVsmE660 challenge.

The administration of plasmid MIP-1α and plasmid Flt3L during initial DNA vaccine priming is next performed to determine whether such administration improves the protective efficacy of the DNA prime/rAd5 boost vaccine regimen in rhesus monkeys with pre-existing anti-Ad5 immunity. An 80% power is estimated to detect a 0.75 log difference in peak viral RNA and a 1.5-2.0 log difference in setpoint viral RNA with 6 monkeys per group. If plasmid MIP-1α and plasmid Flt3L augment vaccine-elicited immune responses, then monkeys in Group 3 may demonstrate more effective control of viral replication than monkeys in Group 2. Such an outcome strongly supports the hypothesis that increasing both recruitment and activation or maturation of professional APCs at the site of inoculation during initial DNA vaccine priming improves the protective efficacy of DNA prime/rAd5 boost vaccine regimens in animals with pre-existing anti-vector immunity. Such a result also demonstrates the potential practical utility of this vaccine strategy and provides a rationale for considering the advancement this strategy into phase I clinical trials.

Alternatively, clear differences in immune responses may be present prior to the viral challenge, but these will in fact fail to improve protective efficacy. This may result from the heterogeneity in outcomes following SIVsmE660 infection and the small numbers of monkeys proposed in this study. If no differences in immune responses are observed between Groups 2 and 3 prior to the viral challenge, then no differences in protective efficacy are expected to emerge following challenge. If this occurs, then this study still yields valuable data regarding the ability of pre-existing anti-Ad5 immunity to inhibit the immunogenicity of rAd5 vaccines in rhesus monkeys. Taken together, this study determines the extent to which the protective efficacy of DNA prime/rAd5 boost vaccine strategies is compromised by anti-Ad5 immunity. The inhibitory effects of pre-existing anti-Ad5 immunity may prove to be a major limitation of the rAd5 vaccine candidates currently in large-scale clinical trials, and thus it is important to develop models to study these effects in nonhuman primates. The results of this experiment may therefore yield important data even if the cytokine augmentation strategies unexpectedly prove ineffective.

### EXAMPLE 11

### Prevention and Treatment of Horizontal Transmission of HIV

Patients who are at risk of being infected with the HIV virus can be immunized with the vaccine disclosed by the present invention. High-risk patients include individuals who have been, or will be in contact with the HIV virus, either by blood, or sexual contact. Such patients are thus provided with the vaccine protocol according to the methods of the present invention. Initially, patients are primed with a vaccine regimen comprising four plasmids: a DNA vaccine encoding the HIV gag-pol-nef genes, a second DNA vaccine encoding the HIV env gene, a plasmid encoding Flt3L and a plasmid encoding MIP-1α. 5 mg of each vaccine are administered intramuscularly within the same local area, either in the leg or arm of the patient, as soon as the risk for HIV infection is determined. The four plasmids may be formulated together and co-injected in any combination. Within 2-6 months, patients are boosted intramuscularly with either the same DNA vaccines or with 2×10¹⁰ pfu rAd5-env and 10¹⁰ pfu rAd5-gag-pol-nef. The boost shot may be provided to the patient with or without the Flt3L and MIP-1α adjuvant combination. The immunogenicity of each of these two-injection vaccination regimen may be determined by assessing vaccine-elicited immune responses following primary immunization using the ELISPOT assay, the tetramer binding assay, cytotoxicity assays, lymphoproliferation and antibody ELISA. Optionally, patients may also be administered with a second therapeutic regimen used for HIV, including for example a highly active anti-retroviral therapy (HAART), before, during, or after receiving the vaccine of this invention.

### Example 12

### Accelerated Vaccination Protocol for the Rapid Induction of Immunity in Neonates

The goal of a pediatric AIDS vaccine is to prevent cases of vertical HIV-1 transmission that can occur during the postnatal period as a result of breastfeeding. In light of the fact that neonates tend to have weaker immune systems than adults, an effective pediatric AIDS vaccine should induce potent immune responses in neonates and provide protection against oral viral challenges. Such a vaccine would also have to elicit a rapid immune response in neonates, since approximately 75% of postnatal HIV-1 transmission occurs within the first 6 months of life. However, typical vaccine regimens that have been developed for adults consist of 3-6 immunizations over a 6-10 month time frame and would therefore not be optimal for use in neonates.

The current invention provides methods to induce an accelerated vaccination protocol for the rapid induction of immunity in neonates. These methods involve substantially increasing the immunogenicity of a vaccine by the administration of Flt3L and MIP-3α-augmented DNA vaccines followed by rAd5 boosts. A two-injection immunization regimen may consist of a single DNA vaccine prime followed as rapidly as possible by a single rAd5 boost. Neonates at 1-2 days of age will be provided once by intramuscular injection with the vaccine regimen consisting of at least one immunogen, GM-CSF and MIP-1α. Thus, the vaccine regimen may consist of four plasmids: the HIV-1 env encoding DNA vaccine, the HIV-1 gag-pol-nef encoding DNA vaccine, plasmid Flt3L, and plasmid MIP-3α. Each plasmid is administered at a weight-adjusted dose of 1 mg/kg (maximum dose of 5 mg each). At 8, 4, or preferably 2 weeks of age, mammals will be boosted once by intramuscular injection with an HIV-1 env encoding rAd5 vector and an HIV-1 gag-pol-nef encoding rAd5 vector each at a weight-adjusted dose of 2×10⁹ pfu/kg (maximum of 10¹⁰ pfu each). The immunogenicity of each of these two-injection accelerated vaccination regimens may be determined by assessing vaccine-elicited immune responses weekly for 16 weeks following primary immunization using the ELLIOT assay, the tetramer binding assay, cytotoxicity assays, lymphoproliferation and antibody ELISA. Delivery of the rAd5 too quickly after the initial vaccine regimen may not optimally harness its boosting capability, which presumably depends on established DNA-primed memory responses. The determination of the optimal timing of delivering these vaccine constructs to generate potent immune responses as rapidly as possible in neonates can be readily determined by one skilled in the art.

### EXAMPLE 13

### Prevention and Treatment of Chronic Myelogenous Leukemia

Since the methods of the present invention may be used for the prevention or treatment of cancer of any type or at any stage of development, a patient at risk or diagnosed with Chronic Myelogenous Leukemia is amenable to treatment according to this invention. Alternatively, patients in remission may also be vaccinated to prevent reoccurrences of cancer. Optionally, the patient may also be treated with other relevant anti-neoplastic therapies, including for example, radiotherapy, chemotherapy, or treatment with Gleevec/STI-571, before, during or after vaccination. The patient may be vaccinated with a vaccine regimen comprising a DNA vaccine encoding at least one immunogen substantially identical to the BCR-Abl oncogene, or alternatively any other misexpressed tumor-associated immunogen, a plasmid encoding Flt3L and a plasmid encoding MIP-1α. Within the next 2 to 6 months, the cancer patient may be boosted with a rAd5 vaccine encoding the same immunogen, or alternatively another tumor-associated immunogen, with or without the Flt3L and MIP-1α plasmids. T cell response may be monitored using the same methods as described above.

### Other Embodiments

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of enhancing the immune response to an immunogen in a mammal, said method comprising providing to said mammal
(i) said immunogen; Flt3L or a biologically active fragment thereof; and MIP-1α, MIP-3α or a biologically active fragment thereof, or
(ii) at least one nucleic acid molecule encoding at least one of (a) said immunogen; (b) Flt3L or a biologically active fragment thereof; and (c) MIP-1α, MIP-3α, or a biologically active fragment thereof; and each polypeptide of (a), (b), or (c) not encoded by said at least one nucleic acid molecule.

2-4. (canceled)

5. The method of claim 1, wherein said Flt3L and said MIP-1α or MIP-3α are provided in a therapeutically effective amount to augment a T cell response in said mammal, wherein said T cell response is a CD4+ T cell response, a CD8+ T cell response, or both.

6. (canceled)

7. The method of claim 5, wherein said T cell response is augmented by at least 20% relative to an untreated control.

8. The method of claim 7, wherein said T cell response is augmented by at least 40% relative to an untreated control.

9. The method of claim 1, wherein said Flt3L, said MIP-1α, or said MIP-3α polypeptide or biologically active fragment thereof is a human, mouse, rat, or monkey polypeptide.

10-11. (canceled)

12. The method of claim 1, wherein said Flt3L, said MIP-1α, or said MIP-3α polypeptide is a full length polypeptide.

13-14. (canceled)

15. The method of claim 1 further comprising a step of administering an additional adjuvant to said mammal.

16. The method of claim 15, wherein said adjuvant is GM-CSF or a biologically active fragment thereof.

17. The method of claim 1, wherein at least two immunogens are provided to said mammal.

18. (canceled)

19. The method of claim 1, wherein said mammal is a human.

20. The method of claim 1, wherein said mammal is a neonate.

21. The method of claim 20, wherein said method is to prevent viral transmission during breastfeeding.

22. The method of claim 1, wherein said method is used to treat or prevent a microbial infection.

23. The method of claim 22 further comprising administering a second anti-microbial therapeutic.

24. The method of claim 23, wherein said second therapeutic is administered within one week of said providing.

25. The method of claim 22, wherein said microbial infection is bacterial, viral, fungal, or parasitic.

26. The method of claim 25, wherein said viral infection is an HIV infection.

27. The method of claim 22, wherein said immunogen is substantially identical to is an antigen associated with said microbial infection.

28. The method of claim 27, wherein said antigen is gp160, p24 VLP, gp41, p31, p55, gp120, Tat, gag, pol, env, nef, rev, or VaxSyn.

29. The method of any one of claim 1, wherein said method is used to treat or prevent autoimmune disease, tissue rejection, or allergic reaction.

30. The method of claim 29 further comprising administering a second therapeutic for treatment of said autoimmune disease, tissue rejection, or allergic.

31. The method of claim 30, wherein said second therapeutic is administered within one week of said providing.

32. The method of claim 29, wherein said immunogen is substantially identical to an antigen associated with said autoimmune disease, tissue rejection, or allergic reaction.

33. The method of claim 1, wherein said method is used to prevent or treat cancer.

34. The method of claim 33 further comprising administering a second anti-cancer therapeutic.

35. The method of claim 34, wherein said second anti-cancer therapeutic is administered within one week of said providing.

36. The method of claim 33, wherein said cancer is selected from the group consisting of melanoma, breast cancer, pancreatic cancer, colon cancer, lung cancer, glioma, hepatocellular cancer, endometrial cancer, gastric cancer, intestinal cancer, renal cancer, prostate cancer, thyroid cancer, ovarian cancer, testicular cancer, liver cancer, head and neck cancer, colorectal cancer, esophagus cancer, stomach cancer, eye cancer, bladder cancer, glioblastoma, and metastatic carcinoma.

37. The method of claim 33, wherein said immunogen is substantially identical to an antigen associated with said cancer.

38. The method of claim 37, wherein said antigen is selected from the group consisting of Melan-A, tyrosinase, p97, β-HCG, GalNAc, MAGE-1, MAGE-2, MAGE-3, MAGE-4, MAGE-12, MART-1, MUC1, MUC2, MUC3, MUC4, MUC18, CEA, DDC, melanoma antigen gp75, Hker 8, high molecular weight melanoma antigen, K19, Tyr1 and Tyr2, members of the pMel 17 gene family, c-Met, PSA, PSM, α-fetoprotein, thyroperoxidase, gp1000, NY-ESO-1, telomerase, C25 colon carcinoma, and p53.

39. (canceled)

40. The method of claim 1, wherein said providing is performed using a single formulation.

41. The method of claim 1, wherein said providing is performed using at least two separate formulations.

42. The method of claim 41, wherein said formulations are provided by the same route of administration.

43. The method of claim 1, wherein said providing is by injection intradermally, intramuscularly, subcutaneously, or intravenously.

44. The method of claim 1, wherein at least one of said nucleic acid molecules is an expression vector comprising a regulatory element operably linked to a polynucleotide sequence encoding any of the polypeptides of (a)-(c).

45. (canceled)

46. The method of claim 44, wherein said expression vector is a viral, a bacterial, or a plasmid vector.

47. The method of claim 46, wherein said viral vector is selected from the group consisting of an adenovirus, a poxvirus, and a lentivirus.

48. The method of claim 44, wherein at least 0.2 ug of expression vector is provided.

49. The method of claim 1 further comprising administering a booster shot to said mammal.

50. The method of claim 49, wherein said booster shot is administered within a year of said providing.

51. The method of claim 49, wherein said booster shot comprises one or more immunogens.

52. The method of claim 49, wherein said booster shot comprises MIP-1α, Flt3L, MIP-3α, or a combination thereof in a therapeutically effective amount.

53. The method of claim 49, wherein said booster shot comprises MIP-1α nd Flt3L; MIP-3α, and Flt3L, or MIP-3α MIP-1α, and Flt3L.

54-55. (canceled)

56. The method of claim 49, wherein said booster shot comprises a recombinant vector comprising a polynucleotide sequence operably linked to regulatory elements encoding said immunogen.

57. The method of claim 56, wherein said recombinant vector is a live recombinant vector selected from a group consisting of an adenovirus, a lentivirus, or a poxvirus.

58. The method of claim 57, wherein said poxvirus is modified vaccinia virus Ankara, or fowl pox.

59. The method of claim 56, wherein at least 0.2 ug of said recombinant vector is provided.

60. The method of claim 57, wherein at least 10⁵ pfu of said live recombinant vector is provided.

61. The method of claim 49, wherein said administering of said booster shot results in at least a 2-fold increase in the T cell response in said mammal as compared to the T cell response in a control mammal not provided with said booster shot, wherein said T cell response is a CD4+ T cell response, a CD8+ T cell response, or both.

62. The method of claim 49, wherein said providing and said administering of said booster shot are by the same route of administration.

63. (canceled)

64. The method of claim 49, wherein said booster shot is formulated for injection intradermally, intramuscularly, subcutaneously, or intravenously.
